(19) United States
(12) Patent Application Publication
(10) Pub. No.: US 2020/0188479 A1
(43) Pub. Date: Jun. 18, 2020

(54) APPARATUS AND METHODS FOR ADMINISTRATION OF A NAUSEOGENIC COMPOUND FROM A DRUG DELIVERY DEVICE

(71) Applicant: INTARCIA THERAPEUTICS, INC., Boston, MA (US)

(72) Inventor: Andrew YOUNG, Boston, MA (US)

(21) Appl. No.: 16/491,275

(22) PCT Filed: Mar. 8, 2018
(86) PCT No.: PCT/US2018/021594

§ 371 Date: Sep. 5, 2019

Related U.S. Application Data
(60) Provisional application No. 62/468,399, filed on Mar. 8, 2017.

(51) Int. Cl. A61K 38/16; A61K 38/26; A61K 9/00

(52) U.S. Cl. CPC A61K 38/16; A61K 9/0004; A61K 9/0024; A61K 38/26

(57) ABSTRACT

Provided is a method for treating a subject, comprising contacting the subject with a drug delivery device comprising a nauseogenic compound, wherein the drug delivery device administers the nauseogenic compound to the subject, and the contacting occurs after an administration of the drug delivery device comprising the nauseogenic compound to a human patient population during a first clinical trial; and wherein less than 10% of the human patient population, to whom the drug delivery device comprising the nauseogenic compound was administered, reported having nausea and/or vomiting during the first clinical trial.

### CROSS REFERENCE TO RELATED APPLICATION

This Application claims priority to, and the benefit of, U.S. Provisional Application Ser. No. 62/468,399, filed on Mar. 8, 2017, which is hereby incorporated by reference herein in its entirety.

### SEQUENCE LISTING

The instant application contains a Sequence Listing which has been submitted in ASCII format via EFS-Web and is hereby incorporated by reference in its entirety. Said ASCII copy, created on Mar. 8, 2018, is named ITCA-051_ST25.txt and is 17,957 bytes in size.

### BACKGROUND

Numerous drugs have been developed that fall short of their therapeutic potential because they are nauseogenic to patients. Such therapies subject patients to nausea and/or vomiting and ultimately render them prone to poor treatment adherence that can accompany oral administration (e.g., of small molecules) or periodic self-injections (e.g., of peptides). Poor treatment adherence of nauseogenic peptides, in particular, is exacerbated in patients with so-called “needle-phobia,” a substantial fear of self-injection, and still more so in patients that grow weary of the nausea and vomiting that often follows self-injection. Methods are needed to more effectively administer nauseogenic compounds, mitigate nausea and vomiting, improve treatment adherence and quality of life for patients, and realize the therapeutic potential of otherwise nauseogenic compounds.

### SUMMARY

Applicant has discovered that side effects of nausea and vomiting that have generally been attributed to certain classes of nauseogenic compounds can be mitigated and potentially eliminated by improved administration of such compounds to patients according to methods disclosed herein.

Drugs administered orally or by injection generally undergo rapid absorption phase during which drug concentrations in plasma reach Cₘₐₓ, followed by an elimination phase during which drug concentrations in plasma fall (See FIG. 1A). Before drug concentrations in plasma fall below a minimum effective concentration (MEC) a subsequent dose is administered to maintain plasma concentrations of drug within a therapeutic range. Multiple administered doses yield plasma concentrations of drug exhibiting numerous peaks and troughs as concentrations of the drug periodically rise and fall (See FIGS. 2-8).

Applicant has discovered benefits of administration of certain nauseogenic compounds, such as long-acting nauseogenic peptides, via certain drug delivery devices, particularly implantable osmotic drug delivery devices. Administration of certain long-acting nauseogenic peptides from an implantable osmotic drug delivery device can be configured to provide incremental absorption of the nauseogenic compound so that it slowly and gradually reaches mean steady state concentration (Cₛₛ) in plasma. Further, mean Cₛₛ is steadily maintained without undergoing an elimination phase and thus without incurring substantial peaks and troughs in plasma concentrations (See FIG. 1B). Applicant has further discovered that certain long-acting nauseogenic peptides, having affinity to albumin and prolonged elimination half-lives in humans, are particularly amenable to the disclosed methods of administration via an implantable osmotic drug delivery device.

As explained in greater detail below, nausea and vomiting from administration of certain nauseogenic compounds, particularly from injection of long-acting nauseogenic peptides, can be curtailed or eliminated upon continuous administration from an implantable drug delivery device that (i) provides gradual absorption of the nauseogenic compound, via slow and steady ramp-up, as it reaches and maintains mean steady state concentration (Cₛₛ); (ii) maintains mean Cₛₛ in plasma for weeks, months, one year or longer, substantially free from an elimination phase and thus without incurring substantial peaks and troughs in plasma concentration; and (iii) minimizes, to the extent possible during (i) and (ii), rate of change, particularly positive rate of change, in plasma concentration over time, expressed herein alternatively as d[nauseogenic compound]/dt or d[drug]/dt. In other words, incidence or prevalence of nausea and vomiting can be curtailed when rate of change in plasma concentration of the nauseogenic compound is minimized during course of treatment. For example, nausea and vomiting can be curtailed when positive rate of change in plasma concentration, d[nauseogenic compound]/dt, is held to less than about +2% per hour of the mean steady state concentration (Cₛₛ) of the nauseogenic compound during the course of treatment.

Provided is a method for treating a subject for type-2 diabetes, comprising contacting the subject with an implantable osmotic drug delivery device comprising a long-acting nauseogenic peptide.

Also provided is an apparatus comprising a drug delivery device and a nauseogenic compound, configured to provide, upon being contacted with a subject: administration of a dose of the nauseogenic compound to the subject; wherein during the first 24 hours following initiation of administration, less than or equal to 90% of mean steady state concentration (Cₛₛ) of the nauseogenic compound is attained in the plasma of the subject; and, once Cₛₛ is attained, Cₛₛ of the nauseogenic compound is maintained in the plasma of the subject for at least two weeks.

Further provided is a related method for treating a subject, comprising contacting the subject with a drug delivery device comprising a nauseogenic compound, wherein the drug delivery device administers the nauseogenic compound to the subject, and the contacting occurs after an administration of the drug delivery device comprising the nauseogenic compound to a human patient population during a first clinical trial; and wherein less than 10% of the human patient population, to whom the drug delivery device comprising the nauseogenic compound was administered, reported having nausea and/or vomiting during the first clinical trial.

Further provided is a related method for treating a subject, comprising contacting the subject with a drug delivery device comprising a nauseogenic compound and the drug delivery device administers the nauseogenic compound to the subject, wherein incidence of nausea and/or vomiting is 10% or less during a first clinical trial regarding administration of the drug delivery device comprising a continuous dose of the nauseogenic compound to a first human patient population; and incidence of nausea and/or vomiting is 15% or greater during a second clinical trial regarding administration of an injectable or oral dose of the nauseogenic compound to a second human patient population.

Further provided is a related method for treating a subject, comprising contacting the subject with a drug delivery device comprising a nauseogenic compound and the drug delivery device administers the nauseogenic compound to the subject, wherein incidence of nausea and/or vomiting, reported as a percentage of a first human patient population, during a first clinical trial regarding administration of the drug delivery device comprising a continuous dose of the nauseogenic compound to the first human patient population is reduced by at least 20% relative to incidence of nausea and/or vomiting, reported as a percentage of a second human patient population, during a second clinical trial regarding an administration of an injectable or an oral dose of the nauseogenic compound to the second human patient population.

Also provided is a related method for treating a subject, comprising contacting the subject with a drug delivery device comprising a dose of a nauseogenic compound, wherein the drug delivery device administers the nauseogenic compound to the subject, during the first 24 hours following initiation of administration, less than or equal to 90% of mean steady state concentration (Cₛₛ) of the nauseogenic compound is attained in the plasma of the subject; and once Cₛₛ is attained, Cₛₛ of the nauseogenic compound is maintained in the plasma of the subject for at least two weeks and d[nauseogenic compound]/dt is held to less than about +2% per hour of the mean steady state concentration (Cₛₛ) of the nauseogenic compound.

Additional embodiments are described in greater detail below.

### BRIEF DESCRIPTION OF THE DRAWINGS

FIG. 1A is a plot illustrating human plasma concentrations of a hypothetical drug administered orally or by injection. Shown is a rapid absorption phase during which drug concentrations in plasma reach Cₘₐₓ, followed by an elimination phase during which drug concentrations in plasma fall. Before drug concentrations in plasma fall below a minimum effective concentration (MEC) a subsequent dose is administered to maintain plasma concentrations of drug within a therapeutic range below a minimum toxic concentration (MTC) and above the MEC. See, for example, FIGS. 2-8.

FIG. 1B is a plot illustrating target non-continuous infusion rates for a nauseogenic compound administered via a drug delivery device, estimated to minimize nausea and/or vomiting relative to oral or injectable administration. Ideal ramp-up should be slow, steady and approach a (Cₛₛ) plateau in ˜4 weeks (t_{1/2}˜10 days). Eventual plasma concentrations of the nauseogenic compound may be e.g., 6× initial plasma concentrations.

According to preferred embodiments described herein, such target rates are achieved with certain long-acting nauseogenic peptides via continuous administration of a predetermined rate at a fixed dose from an implantable osmotic drug delivery device, rather than by increasing the provided dosage from low to high. Despite continuous delivery of a sustained dose of the long-acting nauseogenic compound from the implantable osmotic drug delivery device, plasma concentrations of the nauseogenic compound gradually increase to Cₛₛ.

FIG. 2 is a plot illustrating human plasma concentrations of exenatide administered via periodic injection (BID aqueous solution). Daily dosing is made possible by peptidase resistance of exenatide. Tₘₐₓ˜1.3 hours; t_{1/2}˜3.2 hours (by contrast, peptidase prone GLP-1 t_{1/2}˜3.4 min); peak-trough 82% of peak; peak 1.8× mean; d[drug]/dt 62% mean/hour; 40-41% nausea, 13-18% vomiting in 16-30 weeks.

FIG. 3 is a plot illustrating human plasma concentrations of lixisenatide administered via periodic injection (daily aqueous solution). Daily dosing is made possible by peptidase resistance of lixisenatide. Tₘₐₓ˜1.7 hours. t_{1/2}˜3.0 hours; peak-trough 97% of peak; peak 2.4× mean; d[drug]/dt 204% mean/hour; 26% nausea, 11% vomiting in 24 weeks.

FIG. 4 is a plot illustrating human plasma concentrations of liraglutide administered via periodic injection (daily aqueous solution). Daily dosing is made possible by binding of liraglutide to albumin, avoiding clearance by renal filtration. Tₘₐₓ˜12 hours; peak-trough 39% of peak; peak 1.2× mean; d[drug]/dt 11% of mean/hour; nausea 28%, vomiting 11% in 52 weeks.

FIG. 5 is a plot illustrating human plasma concentrations of semaglutide administered via periodic injection (weekly aqueous solution). Daily dosing is made possible by high affinity binding of semaglutide to albumin. Tₘₐₓ˜3.2 days. Weekly dosing is made possible by high albumin affinity, t_{1/2}˜8.3 days; peak-trough 26% of peak; peak 1.12× mean; d[drug]/dt; 3.3% mean/hour; nausea reported 22%, withdrawn 6%.

FIG. 6 is a plot illustrating human plasma concentrations of dulaglutide, albiglutide, and exendin-4 AlbudAb, administered via periodic injection (weekly aqueous solution). Peak-trough: dulaglutide 63% of peak, albiglutide 28% of peak, exendin-4 AlbudAb 31% of peak.

FIG. 7 is a plot illustrating human plasma concentrations of exenatide (Bydureon, poly(lactic-co-glycolic acid (PLGA) encapsulation) following administration of a single bolus injection. Shown is the triphasic release pattern, including a sizeable burst, with maximum release rate ˜2 months. Max d[drug]/dt 63% of mean/hour.

Plasma concentrations reported for a single subcutaneous bolus of exenatide formulated within PLGA matrix (Bydureon) are shown as the symbols. The tri-phasic release comprised an initial burst followed by periods of accelerated release at 2 and 8 weeks after administration. The profile was modeled as the sum of 3 gaussian curves distributed along a logarithmic time domain (X-axis).

FIG. 8 is a plot illustrating human plasma concentrations of exenatide (Bydureon, PLGA encapsulation) administered via periodic injection (weekly aqueous solution). Weekly stacking of triphasic release profiles results in peak-trough 9.9% of peak; peak 1.1× mean; max d[drug]/dt 4.4% of mean/hour; nausea 11.3%, vomiting <5% over 26 weeks. The plasma concentration profile resulting from weekly subcutaneous injections of Bydureon, shown in FIG. 8, were obtained by staggered summation of profiles obtained as described in FIG. 7.

FIG. 9 is a plot illustrating human plasma concentrations of exenatide (non-aqueous formulation) administered via single subdermal placement of an ITCA-650 osmotic drug delivery device. In contrast to human plasma concentrations illustrated in the plots of FIGS. 2-8, the plot of FIG. 9 attains a single peak and does not exhibit peak-trough oscillations in mean plasma concentrations.

FIG. 10 is a summary plot depicting incidence of patients reporting nausea vs. d[drug]/dt for periodic injection of the aqueous formulations of FIGS. 2-8 and for exenatide administered via single subdermal placement of an ITCA-650 osmotic drug delivery device of FIG. 9.

FIG. 11A is a plot estimating mean Cₛₛ over time for liraglutide and semaglutide if administered via single subdermal placement of an osmotic drug delivery device. Comparison is made with mean Cₛₛ over time for exenatide administered via single subdermal placement of an ITCA-650 osmotic drug delivery device (as shown in FIG. 9).

FIG. 11B is a plot comparing estimated d[drug]/dt for exenatide, liraglutide and semaglutide if administered via single subdermal placement of an osmotic drug delivery device. d[semaglutide]/dt is 35× lower than d[exenatide]/dt if administered via single subdermal placement of an osmotic drug delivery device.

FIG. 12 is a summary plot estimating incidence of patients reporting nausea vs. d[drug]/dt for compounds of FIGS. 2-8 and for exenatide of FIG. 9 if each is administered via single subdermal placement of an osmotic drug delivery device.

FIG. 13 is an illustrative model of the pharmacokinetics of subcutaneously (SC) administered GLP-1 agonists. Two compartments (SC and Central) are contemplated. A constant fraction of SC drug enters the Central compartment per unit time (defined by Kₐ). A constant fraction of Central drug is eliminated per unit time (defined by K). Central drug concentration (equal to plasma drug concentration) is an amount of drug in the Central compartment (A) diluted into its volume of distribution (V_{d}).

FIG. 14 is a plot comparing potencies of liraglutide and semaglutide at human GLP-1 receptors based upon final albumin concentration in the incubations. Potencies decreased with increasing albumin concentration, with the mid-range of change occurring with an albumin (HSA) concentration of ˜0.6%.

FIG. 15 shows three plots comparing potency shifts in 4% vs 0.1% albumin, as determined for human GLP-1[7-36]NH₂ (red), liraglutide (blue) and semaglutide (green). There was a small (1.8-fold) increase in potency for human GLP-1 [7-36]NH₂ in 4% albumin. In contrast, there was a 9.3-fold decrease in potency for liraglutide, and a 19.9-fold decrease for semaglutide. Relative to the effect observed with GLP-1[7-36]NH₂, these represent 17.2- and 36.8-fold shifts in potency, respectively, for liraglutide and semaglutide.

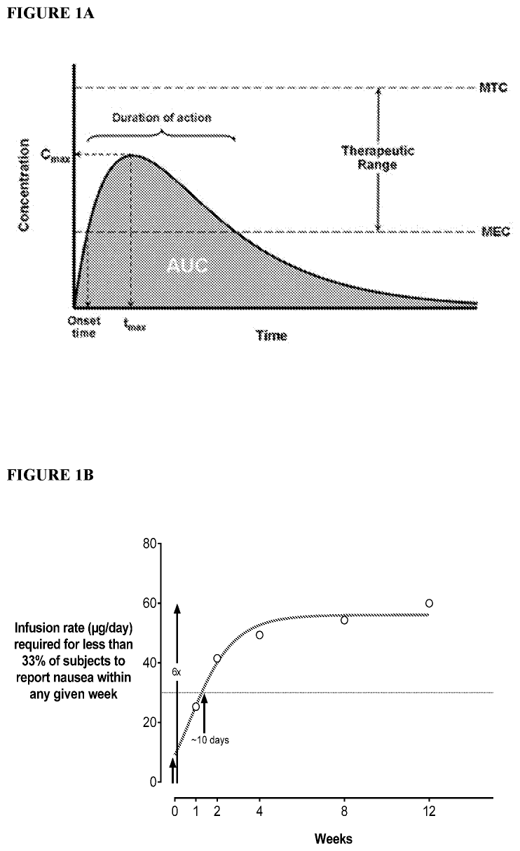

### DETAILED DESCRIPTION

### Definitions

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to “a solvent” includes a combination of two or more such solvents, reference to “a peptide” includes one or more peptides, or mixtures of peptides, reference to “a drug” includes one or more drugs, reference to “an osmotic delivery device” includes one or more osmotic delivery devices, and the like. Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the term “substantially” is understood as within a narrow range of variation or otherwise normal tolerance in the art. Substantially can be understood as within 5%, 4%, 3%, 2%, 1%, 0.5%, 0.1%, 0.05%, 0.01% or 0.001% of the stated value.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

As used herein the term “contacting,” with respect to an implantable drug delivery device refers to subdermal placement or insertion of the implantable drug delivery device, such as an implantable osmotic drug delivery device, beneath a surface of skin of a patient. Alternatively, as used herein the term “contacting,” with respect to an non-implantable drug delivery device, such as a non-implantable miniaturized patch pump, refers to affixing the miniaturized patch pump on an outer surface of skin of a patient.

The terms “peptide,” “polypeptide,” and “protein” are used interchangeably herein and typically refer to a molecule comprising a chain of two or more amino acids (e.g., most typically L-amino acids, but also including, e.g., D-amino acids, modified amino acids, amino acid analogs, and amino acid mimetic). Peptides may be naturally occurring, synthetically produced, or recombinantly expressed. Peptides may also comprise additional groups modifying the amino acid chain, for example, functional groups added via post-translational modification. Examples of post-translation modifications include, but are not limited to, acetylation, alkylation (including, methylation), biotinylation, glutamylation, glycylation, glycosylation, isoprenylation, lipoylation, phosphopantetheinylation, phosphorylation, selenation, and C-terminal amidation. The term peptide also includes peptides comprising modifications of the amino terminus and/or the carboxy terminus. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). The term peptide also includes modifications, such as but not limited to those described above, of amino acids falling between the amino and carboxy termini. In one embodiment, a peptide may be modified by addition of a small-molecule drug. The terms “lower alkyl” and “lower alkoxy” refer to an alkyl or alkoxy group, respectively, having 1-6 carbon atoms.

The term “non-aqueous” as used herein refers to an overall moisture content, for example, of a suspension formulation, typically of less than or equal to about 10 wt %, for example, less than or equal to about 7 wt %, less than or equal to about 5 wt %, and/or less than about 4 wt %. Also, a particle formulation of the present invention comprises less than about 10 wt %, for example, less than about 5 wt %, residual moisture.

The term “implantable delivery device” as used herein typically refers to a delivery device that is fully implanted beneath the surface of a subject's skin to affect administration of a drug.

Representative implantable delivery devices include Hydron® Implant Technology, from Valera Pharmaceuticals. Inc.; NanoGATE™ implant, from iMEDD Inc.; MIP implantable pump or DebioStar™ drug delivery technology, from Debiotech S.A.; Prozor™, Nanopor™ or Delos Pump™, from Delpor Inc.; or an implantable osmotic delivery device, e.g., ITCA-0650, from Intarcia Therapeutics, Inc.

The terms “osmotic delivery device” and “implantable osmotic delivery device” are used interchangeably herein and typically refer to a device used for delivery of a drug (e.g., a nauseogenic compound) to a subject, wherein the device comprises, for example, a reservoir (made, e.g., from a titanium alloy) having a lumen that contains a suspension formulation comprising a drug (e.g., a nauseogenic compound) and an osmotic agent formulation. A piston assembly positioned in the lumen isolates the suspension formulation from the osmotic agent formulation. A semi-permeable membrane is positioned at a first distal end of the reservoir adjacent the osmotic agent formulation and a diffusion moderator (which defines a delivery orifice through which the suspension formulation exits the device) is positioned at a second distal end of the reservoir adjacent the suspension formulation. Typically, the osmotic delivery device is implanted within the subject, for example, subdermally or subcutaneously (e.g., in the inside, outside, or back of the upper arm and in the abdominal area). An exemplary osmotic delivery device is the DUROS® (ALZA Corporation, Mountain View, Calif.) delivery device. Examples of terms synonymous to “osmotic delivery device” include but are not limited to “osmotic drug delivery device”, “osmotic drug delivery system”, “osmotic device”, “osmotic delivery device”, “osmotic delivery system”, “osmotic pump”, “implantable drug delivery device”, “drug delivery system”, “drug delivery device”, “implantable osmotic pump”, “implantable drug delivery system”, and “implantable delivery system”. Other terms for “osmotic delivery device” are known in the art.

The term “continuous delivery” as used herein typically refers to a substantially continuous release of drug from an osmotic delivery device and into tissues near the implantation site, e.g., subdermal and subcutaneous tissues. For example, an osmotic delivery device releases drug essentially at a predetermined rate based on the principle of osmosis. Extracellular fluid enters the osmotic delivery device through the semi-permeable membrane directly into the osmotic engine that expands to drive the piston at a slow and consistent rate of travel. Movement of the piston forces the drug formulation to be released through the orifice of the diffusion moderator. Thus release of the drug from the osmotic delivery device is at a slow, controlled, consistent rate.

Typically, for an osmotic delivery system, the volume of the chamber comprising the drug formulation is between about 100 μl to about 1000 μl, more preferably between about 140 μl and about 200 μl. In one embodiment, the volume of the chamber comprising the drug formulation is about 150 μl.

The terms “substantial steady-state delivery,” “mean steady state concentration” and “Cₛₛ” are used interchangeably herein and typically refers to delivery of a drug at or near a target therapeutic concentration over a defined period of time, wherein the amount of the drug being delivered from an osmotic delivery device is substantially zero-order delivery. Substantial zero-order delivery of an active agent (e.g., a nauseogenic compound) means that the rate of drug delivered is constant and is independent of the drug available in the delivery system; for example, for zero-order delivery, if the rate of drug delivered is graphed against time and a line is fitted to the data the line has a slope of approximately zero, as determined by standard methods (e.g., linear regression).

The term “non-implantable delivery device” as used herein typically refers to a delivery device, including a “non-implantable miniaturized patch pump,” having certain components that are not implanted beneath the surface of a subject's skin to affect administration of a drug.

Representative non-implantable delivery devices (e.g., patch pumps) include Omnipod®, from Insulet Corp.; Solo™, from Medingo; Finesse™, from Calibra Medical Inc.; Cellnovo pump, from Cellnovo Ltd.; CeQur™ device, from CeQur Ltd.; Freehand™, from MedSolve Technologies, Inc.; Medipacs pump, from Medipacs, Inc.; Medtronic pump and MiniMed Paradigm, from Medtronic, Inc.; Nanopump™, from Debiotech S.A. and STMicroelectronics; NiliPatch pump, from NiliMEDIX Ltd.; PassPort®, from Altea Therapeutics Corp.; SteadyMed patch pump, from SteadyMed Ltd.; V-Go™, from Valeritas, Inc.; Finesse, from LifeScan; JewelPUMP™, from Debiotech S.A.; SmartDose Electronic Patch Injector, from West Pharmaceutical Services, Inc.; SenseFlex FD (disposable) or SD (semi-disposable), from Sensile Medical A.G.; Asante Snap, from Bigfoot Biomedical; PicoSulin device, from PicoSulin; and Animas® OneTouch Ping Pump, from Animas Corp.

In some embodiments, the non-implantable miniaturized patch pump is, e.g., JewelPUMP™ (Debiotech S.A.), placed on the surface of the skin. Dosing of the JewelPUMP™ device is adjustable and programmable. As such, mean steady state concentration (Cₛₛ) in plasma of a short-acting nauseogenic compound can gradually be attained, via slow ramp-up of an increasing dosage, in the subject over days, weeks or months. Alternatively, mean steady state concentration (Cₛₛ) in plasma of a long-acting nauseogenic compound can gradually be attained, via slow ramp-up of an increasing dosage and/or via continuous administration of a fixed dose, in the subject over days, weeks or months. The JewelPUMP™ is a miniaturized patch-pump based on a microelectromechanical system (MEMS) with a disposable unit having payload for ultra-precise administration of compound. The disposable unit is filled once with compound and discarded after use, while the controller unit (including the electronics) can be used for 2 years with multiple disposable units. In some embodiments, the JewelPUMP™ is detachable, watertight for bathing and swimming, includes direct access bolus buttons and a discreet vibration & audio alarm on the patch-pump. In some embodiments, the JewelPUMP™ is remotely controlled. In some embodiments, the delivery device is an MEMS-containing non-implantable delivery device, e.g., carried by the patient or placed on the surface of the skin. In some embodiments, the delivery device is an MEMS-containing implantable delivery device.

The phrase “drug half-life” or “t_{1/2}” as used herein refers how long it takes a drug to be eliminated from blood plasma by one half of its concentration. A drug's half-life is usually measured by monitoring how a drug degrades when it is administered via injection or intravenously. A drug is usually detected using, for example, a radioimmunoassay (RIA), a chromatographic method, an electrochemiluminescent (ECL) assay, an enzyme linked immunosorbent assay (ELISA) or an immunoenzymatic sandwich assay (IEMA). In some embodiments, “drug half-life” or “t_{1/2}” refers how long it takes a drug to be eliminated from human blood plasma by one half of its concentration.

The term “serum” is meant to mean any blood product from which a substance can be detected. Thus, the term serum includes at least whole blood, serum, and plasma.

As used herein the term “nauseogenic compound” is meant to mean any compound associated with an incidence of nausea and/or vomiting of greater than or equal to 5% in a patient population during at least one clinical trial (e.g., generally referred to herein as a second clinical trail) regarding treatment of a disorder or disease with the nauseogenic compound. Certain classes of nauseogenic compounds, including nauseogenic peptides, particularly for treatment of type-2 diabetes, are described in greater detail herein. These and other structurally disparate nauseogenic compounds commonly contribute to an incidence of nausea of equal to or greater than 5% in a patient population during at least one clinical trial. In some embodiments, the nauseogenic compound is associated with a higher incidence of nausea and/or vomiting of at least 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or from 10-20%, 20-30%, 30-40%, 40-50%, 50-75%, 75-100%, in a patient population during at least one clinical trial (e.g., second clinical trail) regarding treatment of a disorder or disease with the nauseogenic compound. By contrast, such established nauseogenic compounds, when administered according to disclosed methods, are associated with reduced incidence of nausea and/or vomiting, for example, during treatment or a related clinical trail (e.g., generally referred to herein as a first clinical trail) relative to incidence of nausea and/or vomiting described above in the second clinical trail.

Certain embodiments relate to an incidence of nausea for the nauseogenic compound. Other embodiments relate to an incidence of vomiting for the nauseogenic compound. Some embodiments relate to an incidence of nausea or vomiting for the nauseogenic compound. Some embodiments relate to an incidence of nausea and vomiting for the nauseogenic compound.

The terms “incidence of nausea,” “incidence of vomiting” and “incidence of nausea and/or vomiting” as used herein may refer to a percentage of subjects or patients in a patient population that has experienced nausea and/or vomiting, at least once, during a period of time following subcutaneous administration of a nauseogenic compound. For example, an incidence of nausea of 10% in a patient population of 100 patients during a clinical trial lasting 52 weeks, means that 10 patients experienced nausea at least once during the 52 week period. In some embodiments, incidence of nausea and/or vomiting is determined from the percentage of patients in a patient population who have experienced nausea and/or vomiting, one or more times, throughout the course of a clinical trial, following oral, injectable, or continuous subcutaneous administration via delivery device of a nauseogenic compound. Incidence of nausea and/or vomiting from administration via delivery device, or oral or injectable administration of a nauseogenic compound can be established, e.g., from published clinical studies and/or information provided in the product insert of a marketed nauseogenic compound.

The terms “prevalence of nausea,” “prevalence of vomiting” and “prevalence of nausea and/or vomiting” as used herein may refer to a percentage of subjects or patients in a patient population that has experienced nausea and/or vomiting, at a particular point in time, following subcutaneous administration of a nauseogenic compound. Generally, incidence of nausea and/or vomiting over the course of a clinical trial involves a higher percentage of patients than does prevalence of nausea and/or vomiting at any particular point in time during the clinical trial. In some embodiments, prevalence of nausea and/or vomiting is determined at one or more specific time points following subcutaneous administration. In some embodiments, prevalence of nausea and/or vomiting is determined after a period of time (e.g., 1 week, 1 month, 3 months, 6 months, or 1 year) following subcutaneous administration. Prevalence of nausea and/or vomiting from administration via delivery device, or oral or injectable administration of a nauseogenic compound can be established, e.g., from published clinical studies and/or information provided in the product insert of a marketed nauseogenic compound.

Incidence and prevalence of adverse events, such as nausea and/or vomiting, during a clinical trial is generally reported for a patient population that has been administered a nauseogenic compound, and these results are compared against those for a placebo group that has not been administered the nauseogenic compound. In some preferred embodiments, incidence or prevalence of nausea and/or vomiting, as used herein, is a reported percentage of the patient population regardless of incidence or prevalence of nausea and/or vomiting in a placebo group. In such embodiments, the incidence or prevalence of nausea and/or vomiting in the placebo group is not subtracted from the reported incidence or prevalence of nausea and/or vomiting in the patient population that was administered the nauseogenic compound. In other embodiments, incidence or prevalence of nausea and/or vomiting is a reported percentage of the patient population that was administered the nauseogenic compound minus the incidence or prevalence of nausea and/or vomiting in the placebo group.

As used herein, a “short-acting nauseogenic peptide” such as a “short-acting GLP-1 receptor agonist peptide” is a nauseogenic peptide having an elimination half-life (t_{1/2}) in humans of less than about 5 hours following subcutaneous administration.

As used herein, “long-acting nauseogenic peptide” such as a “long-acting GLP-1 receptor agonist peptide” is a nauseogenic peptide having an elimination half-life (t_{1/2}) in humans of at least about 5 hours following subcutaneous administration. In some embodiments, the nauseogenic peptide has an elimination half-life (t_{1/2}) in humans of at least about 8 hours, 10 hours, 12 hours, 16 hours, 24 hours or longer following subcutaneous administration.

### DESCRIPTION OF EXEMPLARY EMBODIMENTS

Applicant has discovered benefits of administration of certain nauseogenic compounds via a drug delivery device that is configured to (i) provide gradual absorption of the nauseogenic compound, via slow and steady ramp-up, as it reaches and maintains mean steady state concentration (Cₛₛ); (ii) maintains mean Cₛₛ in plasma for weeks, months, one year or longer, substantially free from an elimination phase and thus without incurring substantial peaks and troughs in plasma concentration; and (iii) minimize, to the extent possible during (i) and (ii), rate of change in plasma concentration over time, particularly positive rate of change, expressed herein alternatively as d[nauseogenic compound]/dt or d[drug]/dt. Positive rate of change in plasma concentration over time is maximized by occurrence of sudden spikes (i.e., rate increases) or during peak-trough fluctuations in plasma concentration that are generally attributable to periodic oral or injectable administrations. By contrast, positive rate of change in plasma concentration during treatment is minimized during slow and steady ramp-up of plasma concentration of a nauseogenic compounds in the absence of peak-trough fluctuations, e.g., according to the methods of administration described herein. Benefits of the methods of administration described herein include reduced or eliminated incidence of nausea and/or vomiting for nauseogenic compounds, particularly relative to oral or injectable administration of the same.

Provided in a first aspect is a method for treating a subject for type-2 diabetes, comprising contacting the subject with an implantable osmotic drug delivery device comprising a long-acting nauseogenic peptide. Such methods, without being bound by theory, configure the implantable osmotic drug delivery device and long-acting nauseogenic peptide to (i) provide gradual absorption of the nauseogenic compound, via slow and steady ramp-up, as it reaches and maintains mean steady state concentration (Cₛₛ); (ii) maintain mean Cₛₛ in plasma for weeks, months, one year or longer; and (iii) minimize, to the extent possible during (i) and (ii), rate of change in plasma concentration over time.

Also provided in a second aspect is an apparatus comprising a drug delivery device and a nauseogenic compound, configured to provide, upon being contacted with a subject: administration of a dose of the nauseogenic compound to the subject; wherein during the first 24 hours following initiation of administration, less than or equal to 90% of mean steady state concentration (Cₛₛ) of the nauseogenic compound is attained in the plasma of the subject; and, once Cₛₛ is attained, Cₛₛ of the nauseogenic compound is maintained in the plasma of the subject for at least two weeks.

Further provided in a third aspect is a related method for treating a subject, comprising contacting the subject with a drug delivery device comprising a first dose of a nauseogenic compound, wherein the drug delivery device administers the nauseogenic compound to the subject, and the contacting occurs after an administration of the drug delivery device comprising the nauseogenic compound to a human patient population during a first clinical trial; where less than 10% of the human patient population, to whom the drug delivery device comprising the first dose of the nauseogenic compound was administered, reported having nausea and/or vomiting during the first clinical trial.

Also provided in the third aspect is a nauseogenic compound, for use in a method of treating a subject, comprising contacting the subject with a drug delivery device comprising a first dose of the nauseogenic compound, wherein the drug delivery device administers the nauseogenic compound to the subject, and the contacting occurs after an administration of the drug delivery device comprising the nauseogenic compound to a human patient population during a first clinical trial; wherein less than 10% of the human patient population, to whom the drug delivery device comprising the first dose of the nauseogenic compound was administered, reported having nausea and/or vomiting during the first clinical trial.

As described herein, nauseogenic compounds are associated with a high incidence (e.g., at least 5% but sometimes about 10%-15% or 15%-20% or greater than 20%) of nausea and/or vomiting in a patient population during at least one clinical trial (e.g., generally referred to herein as a second clinical trail) regarding treatment of a disorder or disease with the nauseogenic compound. Methods according to the third aspect reduce the incidence of nausea and/or vomiting (e.g., about 10% or less) in a patient population, e.g., as evidenced during at least one clinical trial regarding administration of drug delivery device comprising the first dose of the nauseogenic compound (e.g., generally referred to herein as a first clinical trail).

In some embodiments, the percentage of human patients who reported having nausea and/or vomiting during the first clinical trial is less than (e.g., 10% to 25% less than, 25% to 50% less than, 50% to 75% less than, 75% to 99% less than) the percentage that reported having nausea and/or vomiting during a second clinical trial regarding an injectable form of the nauseogenic compound.

In some embodiments, the percentage of human patients who reported having nausea and/or vomiting during the first clinical trial is less than (e.g., 10% to 25% less than, 25% to 50% less than, 50% to 75% less than, 75% to 99% less than) the percentage that reported having nausea and/or vomiting during a second clinical trial regarding an orally available form of the nauseogenic compound.

In some embodiments, the drug delivery device delivers the nauseogenic compound to the subject. In other embodiments, the drug delivery device provides the nauseogenic compound to the subject.

Further provided in a fourth aspect is a related method for treating a subject, comprising contacting the subject with a drug delivery device comprising a nauseogenic compound and the drug delivery device administers the nauseogenic compound to the subject, wherein incidence of nausea and/or vomiting is 10% or less during a first clinical trial regarding administration of the drug delivery device comprising a continuous dose of the nauseogenic compound to a first human patient population; and incidence of nausea and/or vomiting is 15% or greater during a second clinical trial regarding administration of an injectable or oral dose of the nauseogenic compound to a second human patient population.

Also provided in the fourth aspect is a nauseogenic compound, for use in a method of treating a subject, comprising contacting the subject with a drug delivery device comprising the nauseogenic compound and the drug delivery device administers the nauseogenic compound to the subject, wherein incidence of nausea and/or vomiting is 10% or less during a first clinical trial regarding administration of the drug delivery device comprising a continuous dose of the nauseogenic compound to a first human patient population; and incidence of nausea and/or vomiting is 15% or greater during a second clinical trial regarding administration of an injectable or oral dose of the nauseogenic compound to a second human patient population.

As explained, nauseogenic compounds are associated with a high incidence (e.g., at least 5% but sometimes about 10%-15% or 15%-20% or greater than 20%) of nausea and/or vomiting in a patient population during at least one clinical trial (e.g., generally referred to herein as a second clinical trail) regarding treatment of a disorder or disease with the nauseogenic compound. Methods according to the fourth aspect reduce the incidence of nausea and/or vomiting (e.g., to about 10% or less) in a patient population, e.g., as evidenced during at least one clinical trial regarding administration of drug delivery device comprising the first dose of the nauseogenic compound (e.g., generally referred to herein as a first clinical trail).

The terms “first clinical trial” and “second clinical trial” are merely used to distinguish clinical trials and do not imply that the “first clinical trial” was conducted prior to the “second clinical trial.” Generally, the “second clinical trial” pertaining to an injectable or oral administration of the nauseogenic compound precedes the “first clinical trial” pertaining to administration with a drug delivery device comprising a nauseogenic compound. Similarly, the terms “first human patient population” and “second human patient population” are merely used to distinguish human patient populations and do not imply that the “first human patient population” was treated or administered the nauseogenic compound prior to administration to the “second human patient population.”

The term, “clinical trial,” as used herein refer to any medical study of a human patient population of between ten and ten thousand patients, at least some of whom have been administered (e.g., orally, via injection or upon continuous subcutaneous administration via delivery device) a nauseogenic compound for the treatment of any disease or disorder such as diabetes, e.g., type-2 diabetes, obesity or any of the “variety of conditions” described herein. The clinical trial is conducted to determine the safety and efficacy for treatment of the disease or disorder in the human patient population upon administration of the nauseogenic compound for a period of time from e.g., weeks to months to years. Generally, clinical trials include at least one “treatment arm” of the human patient population to whom the nauseogenic compound is administered and at least one “placebo arm” to whom a placebo, rather than the nauseogenic compound, is administered.

Continuous dosing via delivery device, injectable dosing, and an oral dosing of the same nauseogenic compound generally differ in the amount of compound that is administered and need not be the same. For example, continuous dosing via delivery device may be e.g., 10 μg/day to 300 μg/day of a nauseogenic compound, injectable dosing may be e.g., 5 μg/injection to 300 μg/injection of the nauseogenic compound, and oral dosing may be e.g. 10 mg/tablet to 3,000 mg/tablet of the nauseogenic compound.

In some embodiments, the dose (e.g., continuous dosing via delivery device) is 10-50 μg/day, 50-100 μg/day, 100-150 μg/day, or 150-300 μg/day. In some embodiments, the dose (e.g., injectable dosing) is 10-50 μg/injection, 50-100 μg/injection, 100-150 μg/injection, or 150-300 μg/injection. In some embodiments, the dose (e.g., oral dosing) is 10-50 mg/tablet, 50-500 mg/tablet, 500-1,000 mg/tablet, 1,000-3,000 mg/tablet.

All such doses and others, in any combination, are applicable to the disclosed methods despite potential differences in absolute amounts of nauseogenic compound that are dosed continuously and subcutaneously via delivery device, orally, or via injection. Rather, the disclosed methods relate to reductions in the incidence of nausea and/or vomiting that accompany a drug delivery device that subcutaneously administers an effective amount of the nauseogenic compound to the subject relative to incidence of nausea and/or vomiting that accompany injectable and oral administration of an effective amount of the nauseogenic compound, regardless of absolute or relative doses administered.

In some embodiments, the second clinical trial relates to an administration of an injectable dose of the nauseogenic compound to a second human patient population. In some embodiments, the second clinical trial relates to an administration of an oral dose of the nauseogenic compound to a second human patient population. In some embodiments, the first clinical trial relates to continuous administration of the nauseogenic compound via delivery device to a human patient population.

Generally, regardless of exact percentages, incidence of nausea and/or vomiting is reduced upon administration of the drug delivery device comprising a continuous dose of the nauseogenic compound, according to disclosed methods (e.g., as evidenced by reported results from a first clinical trial), relative to incidence of nausea and/or vomiting upon administration of an injectable or oral dose of the nauseogenic compound, according to existing methods (e.g., as evidenced by reported results from a second clinical trial).

In some embodiments, incidence of nausea and/or vomiting is 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or lower during the first clinical trial regarding administration of the drug delivery device comprising a continuous dose of the nauseogenic compound to the first human patient population.

In some embodiments, incidence of nausea and/or vomiting is 1%-5%, 5%-10%, 10%-15%, 15%-20%, 20-25% or lower during the first clinical trial regarding administration of the drug delivery device comprising a continuous dose of the nauseogenic compound to the first human patient population.

In some embodiments, incidence of nausea and/or vomiting is 99%, 90%, 80%, 70%, 60%, 50%, 40% 30%, 29%, 28%, 27%, 26%, 24%, 23%, 22%, 21%, 20%, 19%, 18%, 17%, 16%, 15%, 14%, 13%, 12%, 11%, 10%, 9%, 8%, 7%, 6%, 5% or greater during the second clinical trial regarding administration of an injectable or oral dose of the nauseogenic compound to the second human patient population.

In some embodiments, incidence of nausea and/or vomiting is 99%-75%, 75%-50%, 50%-25%, 30-20%, 30-15%, 30-10%, 25-5% or greater during the second clinical trial regarding administration of an injectable or oral dose of the nauseogenic compound to the second human patient population.

Further provided in a fifth aspect is a related method for treating a subject, comprising contacting the subject with a drug delivery device comprising a nauseogenic compound and the drug delivery device administers the nauseogenic compound to the subject, wherein incidence of nausea and/or vomiting, reported as a percentage of a first human patient population, during a first clinical trial regarding administration of the drug delivery device comprising a continuous dose of the nauseogenic compound to the first human patient population, is reduced by at least 20% relative to incidence of nausea and/or vomiting, reported as a percentage of a second human patient population, during a second clinical trial regarding an administration of an injectable or an oral dose of the nauseogenic compound to the second human patient population.

Also provided in the fifth aspect is a nauseogenic compound, for use in a method of treating a subject, comprising contacting the subject with a drug delivery device comprising the nauseogenic compound and the drug delivery device administers the nauseogenic compound to the subject, wherein incidence of nausea and/or vomiting, reported as a percentage of a first human patient population, during a first clinical trial regarding administration of the drug delivery device comprising a continuous dose of the nauseogenic compound to the first human patient population, is reduced by at least 20% relative to incidence of nausea and/or vomiting, reported as a percentage of a second human patient population, during a second clinical trial regarding an administration of an injectable or an oral dose of the nauseogenic compound to the second human patient population.

Methods according to the fifth aspect relate to the percentage by which the incidence of nausea and/or vomiting is reduced by comparison of the lower incidence reported during the first clinical trial relative to the higher incidence reported during the second clinical trial.

For example, incidence of nausea and/or vomiting of 5%, reported as a percentage of a first human patient population, during a first clinical trial regarding administration of the drug delivery device comprising a continuous dose of the nauseogenic compound to the first human patient population is reduced by 50% relative to incidence of nausea and/or vomiting of 10%, reported as a percentage of a second human patient population, during a second clinical trial regarding an administration of an injectable or an oral dose of the nauseogenic compound to the second human patient population.

Similarly, incidence of nausea and/or vomiting of 4%, reported as a percentage of a first human patient population, during a first clinical trial regarding administration of the drug delivery device comprising a continuous dose of the nauseogenic compound to the first human patient population is reduced by 80% relative to incidence of nausea and/or vomiting of 20%, reported as a percentage of a second human patient population, during a second clinical trial regarding an administration of an injectable or an oral dose of the nauseogenic compound to the second human patient population.

In some embodiments, incidence of nausea and/or vomiting during a first clinical trial regarding administration of the drug delivery device comprising a continuous dose of the nauseogenic compound to the first human patient population is reduced by 20%-30%, 30%-40%, 40%-50%, 50%-60%, 70%-80%, 80%-90%, 90%-100%, at least 25%, at least 50%, at least 75% relative to incidence of nausea and/or vomiting during a second clinical trial regarding an administration of an injectable or an oral dose of the nauseogenic compound to the second human patient population.

In some preferred embodiments, incidence of nausea and/or vomiting relates to the incidence reported by the human patient population who was administered the nauseogenic compound, and does not factor incidence of nausea and/or vomiting reported by a placebo group. In some embodiments, incidence of nausea and/or vomiting relates to the incidence reported by the human patient population who was administered the nauseogenic compound minus the incidence of nausea and/or vomiting reported by a placebo group.

Certain embodiments relate to an incidence of nausea. Other embodiments relate to an incidence of vomiting. Some embodiments relate to an incidence of nausea or vomiting. Some embodiments relate to an incidence of nausea and vomiting.

Certain embodiments relate to a prevalence of nausea. Other embodiments relate to a prevalence of vomiting. Some embodiments relate to a prevalence of nausea or vomiting. Some embodiments relate to a prevalence of nausea and vomiting.

In some embodiments, the method is provided for treating diabetes in a subject. In some embodiments, the method is provided for treating type-2 diabetes in a subject.

In some embodiments, the nauseogenic compound is a nauseogenic peptide. In some embodiments, the nauseogenic compound is a long-acting nauseogenic peptide.

In some embodiments, the method is provided for treating a subject for type-2 diabetes, comprising contacting the subject with an implantable osmotic drug delivery device comprising a long-acting nauseogenic peptide. In some embodiments, the long-acting nauseogenic peptide is selected from GLP-1 receptor agonist, PYY analog, amylin agonist, CGRP analog, or neurotensin analog. In some embodiments, the nauseogenic compound is a GLP-1 receptor agonist. In some embodiments, the long-acting GLP-1 receptor agonist is exenatide dispersed in a biocompatible polymer (Bydureon®), semaglutide (Ozempic®), liraglutide (Victoza®), albiglutide (Tanzeum®), or dulaglutide (Trulicity®). In some embodiments, the long-acting GLP-1 receptor agonist is semaglutide. In some embodiments, the long-acting GLP-1 receptor agonist is liraglutide. In some embodiments, the long-acting GLP-1 receptor agonist is albiglutide. In some embodiments, the long-acting GLP-1 receptor agonist is dulaglutide. In some embodiments, the long-acting GLP-1 receptor agonist is exenatide dispersed in a biocompatible polymer.

Further provided in a sixth aspect is a related method for treating a subject, comprising contacting the subject with a drug delivery device comprising a dose of a nauseogenic compound, wherein the drug delivery device administers the nauseogenic compound to the subject, during the first 24 hours following initiation of administration, less than or equal to 90% of mean steady state concentration (Cₛₛ) of the nauseogenic compound is attained in the plasma of the subject; and once Cₛₛ is attained, Cₛₛ of the nauseogenic compound is maintained in the plasma of the subject for at least two weeks.

Also provided is a nauseogenic compound, for use in a method for treating a subject, comprising contacting the subject with a drug delivery device comprising a first dose of the nauseogenic compound, wherein the drug delivery device administers the nauseogenic compound to the subject, during the first 24 hours following initiation of administration, wherein less than or equal to 90% of mean steady state concentration (Cₛₛ) of the nauseogenic compound is attained in the plasma of the subject; and once Cₛₛ is attained, Cₛₛ of the nauseogenic compound is maintained in the plasma of the subject for at least two weeks.

Each of the embodiments described herein relate to any and all aspects of the invention including the first, second and/or third aspect from the preceding paragraph.

In some embodiments, incidence (e.g., mean incidence during a period of time or during a clinical trial) of nausea and/or vomiting is reduced in the subject or in a patient population during treatment with a nauseogenic compound by the present methods, i.e., by administration via drug delivery device, relative to incidence of nausea and/or vomiting from oral or injectable administration of the same nauseogenic compound. Reduced incidence of nausea and/or vomiting may be established and compared against results of incidence of nausea and/or vomiting in pre-clinical studies, including animal models (e.g., reduced appetite in rats or the onset of emesis in dogs) for nausea, vomiting, or reduced food intake. Additionally, incidence of nausea and/or vomiting from oral or injectable administration of a nauseogenic compound can be established, e.g., from published clinical studies and/or information provided in the product insert of a marketed nauseogenic compound.

In some embodiments, prevalence (e.g., statistical prevalence at a given point in time) of nausea and/or vomiting is reduced in the subject or in a patient population during treatment with a nauseogenic compound by the present methods, i.e., by administration via drug delivery device, relative to prevalence of nausea and/or vomiting from oral or injectable administration of the same nauseogenic compound. Prevalence of nausea and/or vomiting may be established and compared against results of pre-clinical studies, including animal models (e.g., reduced appetite in rats or the onset of emesis in dogs) for nausea, vomiting, or reduced food intake. Additionally, prevalence of nausea and/or vomiting from oral or injectable administration of a nauseogenic compound can be established, e.g., from published clinical studies and/or information provided in the product insert of a marketed nauseogenic compound.

In some embodiments, the method for treating the subject includes a dose escalation, further comprising contacting the subject with an additional drug delivery device comprising a second dose of the nauseogenic compound, wherein the second dose is higher than the first dose. In some embodiments, the method for treating a subject does not include a dose escalation, comprising contacting the subject with an additional drug delivery device comprising the first dose of the nauseogenic compound.

In some embodiments, the percentage of the human patient population who reported having nausea and/or vomiting, at the first and/or second dose, during the clinical trials is disclosed in published clinical studies and/or information provided in the product insert (i.e., prescribing information) of a marketed drug delivery device comprising the nauseogenic compound. In some embodiments, the percentage is a mean percentage.

In some embodiments, the percentage of the human patient population who reported having nausea and/or vomiting, at the first and/or second dose, during the clinical trials was less than 20%, 19%, 18%, 17%, 16%, 15%, 14%, 13%, 12%, 11%, 10%, 9%, 8%, 7%, 6%, 5%, 4%, 3%, 2% or 1%. In some embodiments, the percentage of the human patient population who reported having nausea and/or vomiting, at the first and/or second dose, during the clinical trials was 20%, 19%, 18%, 17%, 16%, 15%, 14%, 13%, 12%, 11%, 10%, 9%, 8%, 7%, 6%, 5%, 4%, 3%, 2% or 1%. In some embodiments, less than 15% of the human patient population reported having nausea and/or vomiting, at the first and/or second dose, during the clinical trials. In some embodiments, less than 10% of the human patient population reported having nausea and/or vomiting, at the first and/or second dose, during the clinical trials. In some embodiments, less than 5% of the human patient population reported having nausea and/or vomiting, at the first and/or second dose, during the clinical trials. In some embodiments, from 0.01% to 1% of the human patient population reported having nausea and/or vomiting, at the first and/or second dose, during the clinical trials. In some embodiments, from 0.01% to 2% of the human patient population reported having nausea and/or vomiting, at the first and/or second dose, during the clinical trials. In some embodiments, the percentage of human patient population reported as having nausea and/or vomiting at the first and/or second dose, during the clinical trials ranges from 0.01%-5%, 0.1%-5%, 1%-5%, 0.01%-10%, 0.1%-10%, or 1%-10%.

In some embodiments, the number of patients in the human patient population in the clinical trials who are administered the drug delivery device comprising the first and/or second dose of a nauseogenic compound is from 20 to 1000. In some embodiments, the number of patients is from 20 to 200, 201 to 500, 501 to 1000, 1001 to 2000, 2001 to 3000, or 3001 to 4000.

In some embodiments, patients in the human patient population were treated, on average, for 20 to 200 weeks, 20 to 100 weeks, 20 to 50 weeks, 51 to 100 weeks, or 101-200 weeks with drug delivery device comprising the first and/or second dose of a nauseogenic compound.

In some embodiments, clinical trials include a placebo group of human patients who are not administered the drug delivery device comprising the first and/or second dose of the nauseogenic compound, and an active compound group of human patients who are administered the drug delivery device comprising the first and/or second dose of a nauseogenic compound. In some embodiments, both groups report having nausea and/or vomiting during the clinical trials, from the first and/or second dose of the nauseogenic compound, and the difference between the percentage of human patients in the active compound group who report having nausea and/or vomiting, and the percentage of human patients in the placebo group who report having nausea and/or vomiting, is less than 15%, 14%, 13%, 12%, 11%, 10%, 9%, 8%, 7%, 6%, 5%, 4%, 3%, 2% or 1%. In some embodiments, the percentage of human patients in the active compound group who report having nausea and/or vomiting, from the first and/or second dose of the nauseogenic compound, is higher than the percentage of human patients in the placebo group who report having nausea and/or vomiting. In some embodiments, the percentage of human patients in the active compound group who report having nausea and/or vomiting, from the first and/or second dose of the nauseogenic compound, is substantially similar to the percentage of human patients in the placebo group who report having nausea and/or vomiting.

In some embodiments, the percentage of human patients who report having nausea and/or vomiting, from the first and/or second dose of the nauseogenic compound provided by the drug delivery device disclosed herein, is less than the percentage of other human patients that reported having nausea and/or vomiting during previous clinical trials of an injectable form of the nauseogenic compound. In some embodiments, the percentage of human patients who reported having nausea and/or vomiting, from the first and/or second dose of the nauseogenic compound provided by the drug delivery device disclosed herein, was less than the percentage of other human patients that reported having nausea and/or vomiting during previous clinical trials of an orally available form of the nauseogenic compound.

Certain embodiments relate to human patients that reported having nausea from the nauseogenic compound. Other embodiments relate to human patients that reported vomiting from the nauseogenic compound. Other embodiments relate to human patients that reported nausea or vomiting from the nauseogenic compound. Other embodiments relate to human patients that reported nausea and vomiting from the nauseogenic compound.

In certain embodiments, the nauseogenic compound is semaglutide. In certain embodiments, the nauseogenic compound is liraglutide. In certain embodiments, the nauseogenic compound is dulaglutide.

In certain embodiments, the nauseogenic compound is semaglutide and the method is provided for treating type-2 diabetes in the subject. In certain embodiments, the nauseogenic compound is liraglutide and the method is provided for treating type-2 diabetes in the subject. In certain embodiments, the nauseogenic compound is dulaglutide and the method is provided for treating type-2 diabetes in the subject.

In certain embodiments, provided is a method for treating type-2 diabetes in the subject, comprising contacting the subject with a drug delivery device comprising a first dose of semaglutide the drug delivery device administers the semaglutide to the subject, and contacting occurs after an administration of the drug delivery device comprising semaglutide to a human patient population during clinical trials; where less than 15% of the human patient population, to whom the drug delivery device comprising the first dose of semaglutide was administered, reported having nausea during the clinical trials. In some embodiments, less than 14%, 13%, 12%, 11%, 10%, 9%, 8%, 7%, 6%, 5%, 4%, 3%, 2% or 1% of the human patient population, to whom the drug delivery device comprising the first dose of semaglutide was administered, reported having nausea during the clinical trials.

In certain embodiments, provided is a method for treating type-2 diabetes in the subject, comprising contacting the subject with a drug delivery device comprising a first dose of liraglutide, wherein the drug delivery device administers the liraglutide to the subject, and the contacting occurs after an administration of the drug delivery device comprising semaglutide to a human patient population during clinical trials; where less than 15% of the human patient population, to whom the drug delivery device comprising the first dose of liraglutide was administered, reported having nausea during the clinical trials. In some embodiments, less than 14%, 13%, 12%, 11%, 10%, 9%, 8%, 7%, 6%, 5%, 4%, 3%, 2% or 1% of the human patient population, to whom the drug delivery device comprising the first dose of liraglutide was administered, reported having nausea during the clinical trials.

In certain embodiments, provided is a method for treating type-2 diabetes in the subject, comprising contacting the subject with a drug delivery device comprising a first dose of dulaglutide, wherein the drug delivery device administers the dulaglutide to the subject, and the contacting occurs after an administration of the drug delivery device comprising dulaglutide to a human patient population during clinical trials; where less than 15% of the human patient population, to whom the drug delivery device comprising the first dose of dulaglutide was administered, reported having nausea during the clinical trials. In some embodiments, less than 14%, 13%, 12%, 11%, 10%, 9%, 8%, 7%, 6%, 5%, 4%, 3%, 2% or 1% of the human patient population, to whom the drug delivery device comprising the first dose of dulaglutide was administered, reported having nausea during the clinical trials.

In certain embodiments, the nauseogenic compound is a nauseogenic peptide selected from the group consisting of adrenomedullin, amylin, angiotensin II, atrial natriuretic peptide, cholecystokinin, chorionic gonadotropin leuteinizing hormone, corticotrophin releasing factor, endothelins, gastrin, ghrelin, glucagon, glucagon-like peptide 1 (GLP-1), insulin, insulin-like growth factor, leptin, leu-enkephalin, melanocortins, neurotensin, oxytocin, parathyroid hormones (e.g., PTH, PTHrP), pituitary adenylate cyclase activating peptide (PACAP), prolactin, prolactin releasing peptide, somatostatin, tachykinins (e.g., substance P), thyrotropin releasing hormone, vasoactive intestinal peptide (VIP), vasopressin, neuropeptide Y (NPY), pancreatic polypeptide (PP) and peptide YY (PYY), and an agonist thereof or an agonist of the receptor thereof.

In some embodiments, the method is provided for treatment of type-2 diabetes in the subject. In some embodiments, the method is provided for providing glycemic control in the subject. In some embodiments, the method is provided for treatment (including e.g., prevention, inhibition, suppression, delaying the progression) of a “variety of conditions” in the subject, wherein “variety of conditions,” as used herein, includes but is not limited to the following: chronic pain, hemophilia and other blood disorders, endocrine disorders, metabolic disorders, non-alcoholic fatty liver disease (NAFLD), non-alcoholic steatohepatitis (NASH), Alzheimer's disease, cardiovascular diseases (e.g., heart failure, atherosclerosis, and acute coronary syndrome), rheumatologic disorders, diabetes (including type 1, type 2 diabetes mellitus, human immunodeficiency virus treatment-induced, latent autoimmune diabetes in adults, and steroid-induced), obesity, hypoglycemia unawareness, restrictive lung disease, chronic obstructive pulmonary disease, lipoatrophy, metabolic syndrome, leukemia, hepatitis, renal failure, infectious diseases (including bacterial infection, viral infection (e.g., infection by human immunodeficiency virus, hepatitis C virus, hepatitis B virus, yellow fever virus, West Nile virus, Dengue virus, Marburg virus, and Ebola virus), and parasitic infection), hereditary diseases (such as cerebrosidase deficiency and adenosine deaminase deficiency), hypertension, septic shock, autoimmune diseases (e.g., Grave's disease, systemic lupus erythematosus, multiple sclerosis, and rheumatoid arthritis), shock and wasting disorders, cystic fibrosis, lactose intolerance, Crohn's diseases, inflammatory bowel disease, gastrointestinal cancers (including colon cancer and rectal cancer, breast cancer, leukemia, lung cancer, bladder cancer, kidney cancer, non-Hodgkin lymphoma, pancreatic cancer, thyroid cancer, endometrial cancer, and other cancers). Further, some of the above agents are useful for the treatment of infectious diseases requiring chronic treatments including, but not limited to, tuberculosis, malaria, leishmaniasis, trypanosomiasis (sleeping sickness and Chagas disease), and parasitic worms.

In some embodiments, the method for treatment of the subject corresponds to the method for treatment of the human patient population during clinical trials. In some embodiments, the subject of the method and the human patient population of the clinical trials are treated for the same condition.

In some embodiments, the drug delivery device administers the nauseogenic compound to the subject, during the first 24 hours following initiation of administration, wherein less than or equal to 90% of mean steady state concentration (Cₛₛ) of the nauseogenic compound is attained in the plasma of the subject; and once Cₛₛ is attained, Cₛₛ of the nauseogenic compound is maintained in the plasma of the subject for at least two weeks.

In some embodiments, during treatment of a patient with a nauseogenic compound by the present methods, the incidence of nausea and/or vomiting is less than 75%, 50%, 25%, 20%, 10%, 5%, 2% or 1% relative to incidence of nausea from oral or injectable administration of the same nauseogenic compound. In some embodiments, during treatment of a patient with a nauseogenic compound by the present methods, the incidence of nausea and/or vomiting is substantially eliminated.

According to the present methods of administration, Cₛₛ of the nauseogenic compound is gradually attained in plasma. For example, in some embodiments, 90% of mean steady state concentration (Cₛₛ) in plasma of the nauseogenic compound is not reached in the subject until 1 week to 8 weeks following administration. In some embodiments, 90% of mean steady state concentration (Cₛₛ) in plasma of the nauseogenic compound is not reached in the subject until 1 week, 2 weeks, 3 weeks, 4 weeks, 5 weeks, 6 weeks, 7 weeks, 8 weeks, or between any two of these time periods, following administration.

In some embodiments, less than or equal to 90% of mean steady state concentration (Cₛₛ) of the nauseogenic compound is attained in the plasma of the subject during the first 36 hours, 48 hours, 60 hours, 72 hours, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 4 weeks, 5 weeks, 6 weeks, 7 weeks, 8 weeks, or between any two of these time periods, following administration.

In some embodiments, an initial concentration (C_{I}) in plasma of the nauseogenic compound, following initiation of administration, is lower than subsequent Cₛₛ, gradually attained. In some embodiments, a maximum steady state concentration Cₘₐₓ of nauseogenic compound does not substantially exceed the mean steady state concentration (Cₛₛ) of the nauseogenic compound.

In some embodiments, initial concentration (C_{I}) in plasma of the nauseogenic compound, during the first 12 hours following initiation of administration, is less than or equal to 50%, 25% or 10% of mean steady state concentration (Cₛₛ) in plasma of the nauseogenic compound that will be attained in the subject. In some embodiments, initial concentration (C_{I}) in plasma of the nauseogenic compound, during the first 24 hours following initiation of administration, is less than or equal to 50%, 25% or 10% of mean steady state concentration (Cₛₛ) in plasma of the nauseogenic compound that will be attained in the subject. In some embodiments, initial concentration (C_{I}) in plasma of the nauseogenic compound, during the first 2 days, 3 days, 4 days, 5 days, 6 days or 7 days following initiation of administration, is less than or equal to 50%, 25% or 10% of mean steady state concentration (Cₛₛ) in plasma of the nauseogenic compound that will be attained in the subject.

Without being bound by theory, peak-trough fluctuations in plasma concentration of the nauseogenic compound, particularly large rates of change in concentration over short periods of time, large d[nauseogenic compound]/dt, have been found to exacerbate the incidence and/or prevalence of nausea and vomiting. In some embodiments, Cₛₛ of the nauseogenic compound is attained in the plasma of the subject without incurring substantial peak-trough fluctuations in plasma concentration of the nauseogenic compound. As referred to herein, a “substantial peak-trough fluctuation” includes fluctuations of at least 1%, 2%, 3%, 4%, 5%, 10%, 20% or 30% relative to Cₛₛ of the nauseogenic compound.

In some embodiments, Cₛₛ, once attained is steadily maintained in plasma of the patient for at least 2 weeks. In some embodiments, Cₛₛ, once attained is steadily maintained in plasma of the patient for 2-6 weeks, 6-10 weeks, 10-14 weeks, or 14-18 weeks. In some additional embodiments, Cₛₛ, once attained is steadily maintained in plasma of the patient for weeks, months, one year or longer. In some embodiments, Cₛₛ, once attained is steadily maintained for one month, two months, three months, four months, five months, six months, nine months, one year, eighteen months, two years or three years. In some embodiments, Cₛₛ is maintained if mean Cₛₛ does not spike or fall within 30%, 20%, 10%, 5%, 2% or 1% during a given period of time.

It has been discovered that nausea and vomiting can be curtailed when rate of change, particularly positive rate of change, in plasma concentration of the nauseogenic compound is minimized during treatment. For example, nausea and/or vomiting can be curtailed when rate of change in plasma concentration, described herein as d[nauseogenic compound]/dt, is held to less than about +5%, +4%, +3%, or +2% per hour relative to the mean steady state concentration (Cₛₛ) of the nauseogenic compound. In other words, mean Cₛₛ is gradually attained based on a rate of change in plasma concentration less than about +5%, +4%, +3%, or +2% per hour. This implies a slow and steady ramp up in concentration of the nauseogenic compound to mean Cₛₛ without substantial fluctuations/changes in concentration over time.

In some embodiments, d[nauseogenic compound]/dt is less than +1% of the mean Cₛₛ of the nauseogenic compound per hour. In some embodiments, d[nauseogenic compound]/dt is less than +0.5% of the mean steady state concentration (Cₛₛ) of the nauseogenic compound per hour. In some embodiments, d[nauseogenic compound]/dt is less than +0.25% of the mean steady state concentration (Cₛₛ) of the nauseogenic compound per hour.

In some embodiments, (i) d[nauseogenic compound]/dt is less than +5%, +4%, +3%, +2%, +1%, +0.5% or +0.25% of the mean Cₛₛ of the nauseogenic compound per hour and (ii) less than or equal to 90% of mean steady state concentration (Cₛₛ) of the nauseogenic compound is attained in the plasma of the subject during the first 36 hours, 48 hours, 60 hours, 72 hours, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 4 weeks, 5 weeks, 6 weeks, 7 weeks, 8 weeks, or between any two of these time periods, following administration.

In some embodiments, d[nauseogenic compound]/dt is less than +4% of the mean steady state concentration (Cₛₛ) of the nauseogenic compound per hour; and less than or equal to 90% of mean steady state concentration (Cₛₛ) of the nauseogenic compound is attained in the plasma of the subject during the first 7 days following administration.

In some embodiments, (i) d[nauseogenic compound]/dt is less than +1% of the mean Cₛₛ of the nauseogenic compound per hour and (ii) less than or equal to 90% of mean steady state concentration (Cₛₛ) of the nauseogenic compound is attained in the plasma of the subject during the first 36 hours, 48 hours, 60 hours, 72 hours, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 4 weeks, 5 weeks, 6 weeks, 7 weeks, 8 weeks, or between any two of these time periods, following administration. In some embodiments, (i) d[nauseogenic compound]/dt is less than +1% of the mean Cₛₛ of the nauseogenic compound per hour and (ii) less than or equal to 90% of mean steady state concentration (Cₛₛ) of the nauseogenic compound is attained in the plasma of the subject during the first 14 days following administration. In some embodiments, (i) d[nauseogenic compound]/dt is less than +1% of the mean Cₛₛ of the nauseogenic compound per hour and (ii) less than or equal to 90% of mean steady state concentration (Cₛₛ) of the nauseogenic compound is attained in the plasma of the subject during the first 6 weeks following administration. In some embodiments, (i) d[nauseogenic compound]/dt is less than +1% of the mean Cₛₛ of the nauseogenic compound per hour and (ii) less than or equal to 90% of mean steady state concentration (Cₛₛ) of the nauseogenic compound is attained in the plasma of the subject during the first 8 weeks following administration.

In some embodiments, (i) d[nauseogenic compound]/dt is less than +0.5% of the mean Cₛₛ of the nauseogenic compound per hour and (ii) less than or equal to 90% of mean steady state concentration (Cₛₛ) of the nauseogenic compound is attained in the plasma of the subject during the first 36 hours, 48 hours, 60 hours, 72 hours, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 4 weeks, 5 weeks, 6 weeks, 7 weeks, 8 weeks, or between any two of these time periods, following administration. In some embodiments, (i) d[nauseogenic compound]/dt is less than +0.5% of the mean Cₛₛ of the nauseogenic compound per hour and (ii) less than or equal to 90% of mean steady state concentration (Cₛₛ) of the nauseogenic compound is attained in the plasma of the subject during the first 14 days following administration. In some embodiments, (i) d[nauseogenic compound]/dt is less than +0.5% of the mean Cₛₛ of the nauseogenic compound per hour and (ii) less than or equal to 90% of mean steady state concentration (Cₛₛ) of the nauseogenic compound is attained in the plasma of the subject during the first 6 weeks following administration. In some embodiments, (i) d[nauseogenic compound]/dt is less than +0.5% of the mean Cₛₛ of the nauseogenic compound per hour and (ii) less than or equal to 90% of mean steady state concentration (Cₛₛ) of the nauseogenic compound is attained in the plasma of the subject during the first 8 weeks following administration.

It has been discovered that nauseogenic compounds having an extended elimination half-life (t_{1/2}) in humans are particularly suitable to the present methods of administration via drug delivery devices. In some embodiments, the nauseogenic compound is a long-acting nauseogenic peptide. In some embodiments, the nauseogenic compound has a t_{1/2} in humans of at least about 1 day, 2 days or 5 days. In some embodiments, the nauseogenic compound has a t_{1/2} in humans of about 1 day to 14 days. In some embodiments, the nauseogenic compound has a t_{1/2} in humans of about 6 days to 14 days. In some embodiments, the nauseogenic compound has a t_{1/2} in humans of about 7 days to 9 days.

Generally speaking, and without being bound by theory, compounds having an extended elimination half-life in humans are amenable to relatively infrequent (e.g., weekly) administration relative to frequent daily or twice-daily administration. Nonetheless, relatively infrequent (e.g., weekly) administration of nauseogenic compounds, although more convenient than daily administration, generally does not address the incidence of adverse events that persists in patients upon administration of the nauseogenic compounds, regardless of the frequency of administration. By contrast, methods disclosed herein mitigate such adverse events, and reduce incidence of nausea in patients, that might otherwise accompany the administration of nauseogenic compounds.

Reduced incidence and/or prevalence of nausea and/or vomiting were found to be most significant for long-acting nauseogenic peptides having affinity to human serum albumin (HSA, alternatively referred to herein as albumin), when administered via drug delivery devices according to the present methods.

In some embodiments, the long-acting nauseogenic peptide, such as an acylated long-acting peptide or acylated long-acting GLP-1 analogue, can simultaneously bind to albumin and its intended receptor, such as the GLP-1 receptor. In some embodiments, the long-acting nauseogenic peptide is an acylated long-acting GLP-1 receptor agonist that bind to the GLP-1 receptor with an affinity below 100 nM, preferable below 30 nM in the presence of 2% albumin.

In some embodiments, the long-acting nauseogenic peptide is an acylated long-acting peptide or acylated long-acting GLP-1 receptor agonist that binds human serum albumin (HSA) and exhibits an albumin-mediated potency decrease (e.g., 10-200 fold, 10-100 fold, 10-50 fold, 10-30 fold, or 10-25 fold) in activation of GLP-1 receptors at 4% HSA versus its potency for activation of GLP-1 receptors at 0.1% HSA. For example, semaglutide exhibits a 19.9× albumin-mediated decrease in potency for activation of GLP-1 receptors at 4% HSA versus its potency for activation of GLP-1 receptors at 0.1% HSA. In some embodiments, the nauseogenic compound is a long-acting nauseogenic peptide having a binding affinity to its intended receptor that is decreased 20-50 fold in the presence of 4% human serum albumin when comparing the binding affinity in the presence of very low concentration 0.1% of human serum albumin.

In some embodiments, the long-acting nauseogenic peptide is an acylated long-acting peptide or acylated long-acting GLP-1 receptor agonist that binds HSA and exhibits a reduction in potency for activation of its intended receptor, such as the GLP-1 receptor in the presence of physiologic concentrations (2-4%) HSA versus the potency observed with low (0.1%) HSA concentrations. By contrast, GLP-1[7-36]NH₂ exhibits no substantial reduction, or a slight increase, in potency for activation of GLP-1 receptors in the presence of physiologic concentrations (2-4%) HSA versus the potency observed with low (0.1%) HAS concentrations.

In some embodiments, the long-acting nauseogenic peptide is an acylated long-acting peptide or acylated long-acting GLP-1 receptor agonist that binds human serum albumin (HSA) and exhibits an albumin-mediated potency shift (e.g., 20-200 fold, 20-100 fold, 20-50 fold, 30-50 fold, or 30-40 fold) relative to any potency shift (e.g., increase or decrease) for human GLP-1[7-36]NH₂. For example, as illustrated by Example 2, GLP-1[7-36]NH₂ exhibits an albumin-mediated 0.54× increase in potency for activation of GLP-1 receptors at 4% HSA versus potency for activation of GLP-1 receptors at 0.1% HSA whereas semaglutide exhibits an albumin-mediated 19.9× decrease in potency for activation of GLP-1 receptors at 4% HSA versus potency for activation of GLP-1 receptors at 0.1% HSA. Semaglutide thus exhibits, in the assay conditions of Example 2, an albumin-mediated potency shift of 36.8-fold (19.9/0.54) relative to potency shift for human GLP-1[7-36]NH₂. In some embodiments, the nauseogenic compound is an acylated long-acting GLP-1 receptor agonist that binds human serum albumin (HSA) and exhibits an albumin-mediated potency decrease 10-25 fold in the presence of 4% human serum albumin relative its potency in the presence of very low concentration 0.1% of human serum albumin.

The term “albumin binding moiety” as used herein means a residue (e.g., aliphatic substituents or acylated group comprising an aliphatic substituent) which permits the long-acting nauseogenic peptide to bind non-covalently to human serum albumin. The long-acting nauseogenic peptide having an attached albumin binding residue typically has an affinity below 10 μM to human serum albumin and preferably below 1 μM. A range of albumin binding residues, having aliphatic substituents, are known including linear and branched lipophilic moieties, described herein, comprising 4-40 carbon atoms.

In some embodiments, the long-acting nauseogenic peptide has an apparent K_{D} for association with albumin not greater than 1 micromole/liter. In some embodiments, the long-acting nauseogenic peptide has an off rate for dissociation of the long-acting nauseogenic peptide from albumin not greater than 0.002/sec. In other words, not more than 0.2% of peptide-albumin complex will dissociate in a drug-free environment in 1 second.

In some embodiments, the long-acting nauseogenic peptide comprises a lipophilic substituent, as described in greater detail below. In some embodiments, the long-acting nauseogenic peptide comprises any one of the lipophilic substituents described in greater detail herein.

In some embodiments, the drug delivery device is an implantable drug delivery device. In some embodiments, the device is an implantable osmotic delivery device.

In some embodiments, the implantable drug delivery device administers a continuous dose of the nauseogenic compound. In some embodiments, treatment consists of a single dose of the nauseogenic compound. In some embodiments, treatment consists of a relatively low initial dose of the nauseogenic compound followed by a higher maintenance dose of the nauseogenic compound. Semaglutide, for example, is administered at a relatively low initial dose of 0.5 mg/week (corresponding to about 71 μg/day) followed by a higher maintenance dose of 1.0 mg/week (corresponding to about 143 μg/day). In some embodiments, the nauseogenic compound is continuously administered at a dose (μg/day) less than, equal to or greater than an FDA-approved maintenance dose (μg/day or mg/week) of the nauseogenic compound administered via bolus injection. In some embodiments, the nauseogenic compound is continuously administered at a dose (μg/day) less than, equal to or greater than an FDA-approved initial dose (μg/day or mg/week) of the nauseogenic compound administered via bolus injection. In some embodiments, the nauseogenic compound is a long-acting nauseogenic peptide. In some embodiments, the long-acting nauseogenic peptide is a long-acting GLP-1 agonist such as semaglutide. In some embodiments, the long-acting nauseogenic peptide is a long-acting GLP-1 agonist such as liraglutide.

In some embodiments, the implantable drug delivery device administers a continuous dose of about 1 mg/day, 500 μg/day, 250 μg/day, 150 μg/day, 143 μg/day, 140 μg/day, 130 μg/day, 120 μg/day, 110 μg/day, 100 μg/day, 90 μg/day, 80 μg/day, 70 μg/day, 60 μg/day, 50 μg/day, 40 μg/day, 30 μg/day, 20 μg/day, 10 μg/day, or a continuous dose between any two of these values, of the nauseogenic compound. In other embodiments, the implantable drug delivery device administers a continuous dose of about 1-10 μg/day, 10-20 μg/day, 20-30 μg/day, 30-40 μg/day, 40-50 μg/day, 50-60 μg/day, 60-70 μg/day, 70-80 μg/day, 90-100 μg/day, 100-110 μg/day, 110-120 μg/day, 120-130 μg/day, 130-140 μg/day, 140-150 μg/day, 150-200 μg/day, 200-250 μg/day, 250-500 μg/day, or 500-1,000 μg/day.

In some embodiments, the device is a non-implantable delivery device. In some embodiments, the device is a non-implantable miniaturized patch pump, e.g., JewelPUMP™ (Debiotech S.A.), placed on the surface of the skin. In some embodiments, dosing of the non-implantable miniaturized patch pump is adjustable and programmable. As such, mean steady state concentration (Cₛₛ) in plasma of a short-acting or long-acting nauseogenic compound can gradually be attained, via slow ramp-up of an increasing dosage, in the subject over days, weeks or months. In some embodiments, the non-implantable miniaturized patch pump is remotely controlled.

In some embodiments, the non-implantable miniaturized patch pump administers a non-continuous dose of the nauseogenic compound. In some embodiments, the non-implantable miniaturized patch pump administers an increasing dose of the nauseogenic compound.

In some embodiments, the non-implantable miniaturized patch pump administers a short-acting nauseogenic peptide. In some embodiments, the non-implantable miniaturized patch pump administers a long-acting nauseogenic peptide.

In some embodiments, a method is provided for treating any condition or disease in a subject, wherein treatment nausea and/or vomiting are side effects of treatment. In some embodiments, a method is provided for treating diabetes in a subject. In some embodiments, a method is provided for treating type-2 diabetes in a subject. In some embodiments, a method is provided for treating obesity in a subject. In some embodiments, a method is provided for effecting weight loss in a subject. In some embodiments, a method is provided for treating cancer in a subject, e.g., by administration of nauseogenic compounds such as chemotherapy. In some embodiments, a method is provided for controlling pain in a subject, e.g., by administration of nauseogenic compounds such as opiates.

In some embodiments, the drug delivery device comprises a solid suspension of the nauseogenic compound. In some embodiments, the drug delivery device comprises a substantially anhydrous formulation of the nauseogenic compound.

Nauseogenic compounds, including nauseogenic peptides, have been developed for the treatment of a variety of diseases and disorders. For example, nauseogenic peptides for the treatment of diabetes, particularly type-2 diabetes (T2D), include glucagon-like peptide-1 (GLP-1) agonists, peptide YY (also known as PYY, peptide tyrosine tyrosine or pancreatic peptide YY₃₋₃₆) analogs, and amylin analogs (e.g., pramlintide, developed by Amylin Pharmaceuticals, marketed by AstraZeneca). GLP-1 agonists, PYY analogs and amylin analogs are administered subcutaneously via periodic self-injections that generally induce nausea in patients.

Such peptides are generally classified as shorter-acting or longer-acting peptides based on their pharmacokinetic (PK) profiles following subcutaneous administration. Regarding GLP-1 agonists, shorter-acting GLP-1 receptor agonists, such as exenatide and lixisenatide (Adlyxin®), have mean terminal half-lives of approximately several hours in human serum, whereas longer-acting GLP-1 receptor agonists, such as liraglutide (Victoza®) and semaglutide, have half-lives in human serum of approximately 16 and 165 hours, respectively, following subcutaneous administration.

In some embodiments, the nauseogenic compound is a nauseogenic peptide selected from GLP-1 receptor agonist, amylin analog, PYY analog (including any of those disclosed in U.S. Patent Application Publication No.: 2014/0329742; said PYY analogs are incorporated herein by reference), amylin agonist, calcitonin gene-related peptide (CGRP) analog, or neurotensin analog.

In some embodiments, the nauseogenic compound is a long-acting nauseogenic peptide selected from GLP-1 receptor agonist, amylin analog, PYY analog, amylin agonist, CGRP analog, or neurotensin analog, each of which comprises a lipophilic group, optionally bound to the peptide via a spacer.

In some embodiments, the nauseogenic compound is a GLP-1 receptor agonist. In some embodiments, the nauseogenic compound is a short-acting GLP-1 receptor agonist. In some embodiments, the nauseogenic compound is a long-acting GLP-1 receptor agonist. In some embodiments, the nauseogenic compound is a GLP-1 receptor agonist co-formulated with insulin. In some embodiments, the nauseogenic compound is a GLP-1 receptor agonist co-formulated with an insulin analog or functional variant.

As used herein, a “functional variant” means a portion of the native protein that preserves the full activity of the native parent protein. In some embodiments, the portion of the native protein preserves partial activity of the native parent protein. In some embodiments, the portion may be part of a complex (protein, carbohydrate, or other). In other embodiments a functional variant is equivalent in meaning to an “analog.”

Insulin analogs, such as those that may be coformulated with a GLP-1 receptor agonist, include ultra-fast rapid-acting insulins (e.g., Novo Nordisk's Fiasp®), rapid-acting insulins (e.g., Lilly's Humalog®, Novo Nordisk's Novolog®, Sanofi's Apidra® or Admelog®), short-acting insulins (e.g., Novo Nordisk's Novolin®) and particularly the long-acting insulins (e.g., insulin detemir, Novo Nordisk's Levemir®; insulin degludec, Novo Nordisk's Tresiba®; or insulin glargine, including Lilly's Basaglar®, Sanofi's Lantus® or Sanofi's Toujeo®). In some embodiments, the GLP-1 receptor agonist is coformulated with an insulin analog that is a long-acting insulin (e.g., insulin detemir, insulin degludec, or insulin glargine).

### Short-Acting GLP-1 Receptor Agonists

Short-acting GLP-1 receptor agonists, as referred to herein, are GLP-1 receptor agonists having a mean terminal half-life in humans of less than 5 hours following subcutaneous administration.

Exenatide (AstraZeneca; Byetta®):

In some embodiments, the short-acting GLP-1 receptor agonist is exenatide. Byetta® was the first approved GLP-1 receptor agonist (in 2005) as antidiabetic therapy for the treatment of T2D. It has a terminal half-life of approximately 2.4 h after subcutaneous administration and is applied twice daily (5 μg & 10 μg per injection). Exenatide has the following amino acid sequence:

(SEQ ID NO: 1)
H-His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-NH₂

Lixisenatide (Sanofi; Adlyxin®)

In some embodiments, the short-acting GLP-1 receptor agonist is lixisenatide, a synthetic analog of exenatide, developed by Zealand Pharma A/S and marketed by Sanofi.

Relative to exenatide, six lysine residues have been added to the C-terminus, which is also amidated, and having one deleted proline residue at the C-terminal region. Lixisenatide, (des-Pro³⁶-exendin-4(1-39)-Lys₆-NH₂), with a mean terminal half-life of approximately 3 h in humans, has the following amino acid sequence, as described in U.S. Patent No.: RE45313:

(SEQ ID NO: 2)
H-His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Lys)₆-NH₂

### Long-Acting GLP-1 Receptor Agonists

Long-acting GLP-1 receptor agonists, as referred to herein, are GLP-1 receptor agonists having a mean terminal half-life in humans of at least 5 hours following subcutaneous administration. In some embodiments, the long-acting GLP-1 receptor agonist has a mean terminal half-life in humans of at least 8, 10, 12, 16, 20, 24 hours, or 2, 3 4, 5, 6, 7 8, 9, 10 or more days following subcutaneous administration.

In some embodiments, the long-acting GLP-1 receptor agonist is exenatide dispersed in a biocompatible polymer (Bydureon®), semaglutide (Ozempic®), liraglutide (Victoza®), albiglutide (Tanzeum®), or dulaglutide (Trulicity®).

In certain embodiments, extensive half-lives of long-acting GLP-1 receptor agonists are attained, at least in part, by (i) slow release of a GLP-1 receptor agonist from polymeric matrices e.g., exenatide extended release Bydureon® (AstraZeneca); (ii) conjugation of a lipophilic substituent to the GLP-1 receptor agonist, e.g., acylated GLP-1 receptor agonists, liraglutide Victoza®; and semaglutide Ozempic®; (both from Novo Nordisk); (iii) conjugation of the GLP-1 receptor agonist to albumin, e.g., albiglutide Tanzeum® (GSK); (iv) conjugation of the GLP-1 receptor agonist to an Fc region of immunoglobulin G (IgG), e.g., dulaglutide Trulicity® (Eli Lilly). Each of these non-limiting representative embodiments is described in greater detail below.

(i) Slow Release of a GLP-1 Receptor Agonist from Polymeric Matrices

Extended Release Exenatide

Bydureon® (developed by Amylin and marketed by AstraZeneca) is a once-weekly formulation of exenatide, in which exenatide is noncovalently sequestered within a biodegradable polymeric matrix microspheres consisting of poly(D,L-lactide-co-glycolide) (PLG). Slow release from the polymeric matrix takes place through diffusion and microsphere breakdown. Exenatide formulated as Bydureon®, for extended release, has the same amino acid sequence (SEQ ID NO:1) as the exenatide of Byetta®. In some embodiments, the long-acting GLP-1 receptor agonist is exenatide dispersed in a biocompatible polymer.

In some embodiments, the long-acting GLP-1 receptor agonist is a pharmaceutical composition comprising exenatide in a biocompatible poly(lactide-co-glycolide) copolymer, as described in U.S. Pat. No. 8,329,648.

In some embodiments, the long-acting GLP-1 receptor agonist is a composition provided for sustained-release of exenatide, consisting essentially of: a biocompatible polymer having dispersed therein about 3%-5% (w/w) exenatide and about 2% (w/w) sucrose, as described in U.S. Pat. No. 7,456,254. In some embodiments, the long-acting GLP-1 receptor agonist is a composition that consists of: a biocompatible polymer having dispersed therein about 5% (w/w) exenatide and about 2% (w/w) sucrose. In some embodiments, the biocompatible polymer is selected from poly(lactides), poly(glycolides), poly(lactide-co-glycolides), poly(lactic acid)s, poly(glycolic acid)s, poly(lactic acid-co-glycolic acid)s and blends and copolymers thereof. In some embodiments, the biocompatible polymer is poly(lactide-co-glycolide) with a lactide:glycolide ratio of about 1:1.

(ii) Conjugation of a Lipophilic Substituent to the GLP-1 Receptor Agonist

Conjugation of one or more “lipophilic substituents” to long-acting nauseogenic peptides, including long-acting GLP-1 receptor agonists, is intended to prolong the action of the long-acting peptide by facilitating binding to serum albumin and delayed renal clearance of the conjugated peptide. As used herein, a “lipophilic substituent” comprises a substituent comprising 4-40 carbon atoms, in particular 8-25 carbon atoms, or 12 to 22 carbon atoms. The lipophilic substituent may be attached to an amino group of the long-acting nauseogenic peptide or long-acting GLP-1 receptor agonist by means of a carboxyl group of the lipophilic substituent which forms an amide bond with an amino group of the amino acid residue to which it is attached. Preferably, the long-acting nauseogenic peptide or long-acting GLP-1 receptor agonists include three, two, or preferably one lipophilic substituent.

In some embodiments, the long-acting nauseogenic peptide or long-acting GLP-1 agonist has only one lipophilic substituent which substituent comprises an alkyl group or a group which has an ω-carboxylic acid group and is attached to the N-terminal amino acid residue of the parent peptide. In some embodiments, the long-acting nauseogenic peptide or long-acting GLP-1 receptor agonist has only one lipophilic substituent which substituent is an alkyl group or a group which has an ω-carboxylic acid group and is attached to the C-terminal amino acid residue of the parent peptide. In some embodiments, the long-acting nauseogenic peptide or long-acting GLP-1 derivative has only one lipophilic substituent which substituent can be attached to any one amino acid residue which is not the N-terminal or C-terminal amino acid residue of the parent peptide.

In some embodiments, the long-acting nauseogenic peptide or long-acting GLP-1 receptor agonist includes two three or four lipophilic substituents. In some embodiments, the lipophilic substituent has a group which can be negatively charged. One preferred such group is a carboxylic acid group. In some embodiments, the lipophilic substituent is a straight-chain or branched alkyl group. In some embodiments, the lipophilic substituent is the acyl group of a straight-chain or branched fatty acid.

In some embodiments, the lipophilic substituent is an acyl group of the formula CH₃(CH₂)ₙCO—, wherein n is an integer from 4 to 38, preferably an integer from 4 to 24, more preferably CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— or CH₃(CH₂)₂₂CO—.

In some embodiments, the lipophilic substituent is an acyl group of a straight-chain or branched alkane α,ω-dicarboxylic acid.

In some embodiments, the lipophilic substituent is an acyl group of the formula HOOC(CH₂)ₘCO—, wherein m is an integer from 4 to 38, preferably an integer from 4 to 24, more preferably HOOC(CH₂)₁₄CO—, HOOC(CH₂)₁₆CO—, HOOC(CH₂)₁₈CO—, HOOC(CH₂)₂₀CO— or HOOC(CH₂)₂₂CO—.

In some embodiments, the lipophilic substituent is attached, optionally via a spacer, to the ε-amino group of a Lys residue contained in the parent peptide of the long-acting nauseogenic peptide or long-acting GLP-1 derivative.

In some embodiments, the lipophilic substituent is attached to the parent peptide of the long-acting nauseogenic peptide or long-acting GLP-1 receptor agonist by means of a “spacer” which is an unbranched alkane α,ω-dicarboxylic acid group having from 1 to 7 methylene groups, preferably two methylene groups which spacer forms a bridge between an amino group of the parent peptide and an amino group of the lipophilic substituent.

In some embodiments, the spacer is an amino acid, for example, succinic acid, Lys, Glu or Asp, or a dipeptide such as Gly-Lys. In some embodiments, where the spacer is succinic acid, one carboxyl group thereof may form an amide bond with an amino group of the amino acid residue, and the other carboxyl group thereof may form an amide bond with an amino group of the lipophilic substituent. In some embodiments, when the spacer is Lys, Glu or Asp, the carboxyl group thereof may form an amide bond with an amino group of the amino acid residue, and the amino group thereof may form an amide bond with a carboxyl group of the lipophilic substituent. In some embodiments, when Lys is used as the spacer, a further spacer may in some instances be inserted between the ε-amino group of Lys and the lipophilic substituent. In one such embodiment, such a further spacer is succinic acid which forms an amide bond with the ε-amino group of Lys and with an amino group present in the lipophilic substituent. In another such embodiment such a further spacer is Glu or Asp which forms an amide bond with the ε-amino group of Lys and another amide bond with a carboxyl group present in the lipophilic substituent, that is, the lipophilic substituent is a Nε-acylated lysine residue. Other preferred spacers are Nε-(γ-L-glutamyl, Nε-(β-L-asparagyl), Nε-glycyl, and Nε-(α-(γ-aminobutanoyl). In some embodiments, the lipophilic substituent has a group which can be negatively charged, for example, a carboxylic acid group or other compound that has a carboxyl group.

Representative long-acting GLP-1 agonists comprising a single lipophilic substituent include liraglutide and semaglutide.

Liraglutide

(Victoza®, developed and marketed by Novo Nordisk) is administered via daily injection for treatment of type-2 diabetes. Liraglutide has 97% sequence identity to GLP-1(7-37). Liraglutide is modified by two amino acid changes (one addition and one substitution) and by the addition of a lipophilic substituent that enables it to form a noncovalent bond with serum albumin following subcutaneous administration. In some embodiments, the long-acting GLP-1 receptor agonist is liraglutide, i.e., Lys²⁶(N^{ε}-(γ-glutamyl(N^{α}-hexadecanoyl))), Arg³⁴-GLP-1(7-37), which has the following structural Formula I (SEQ ID NO:3):

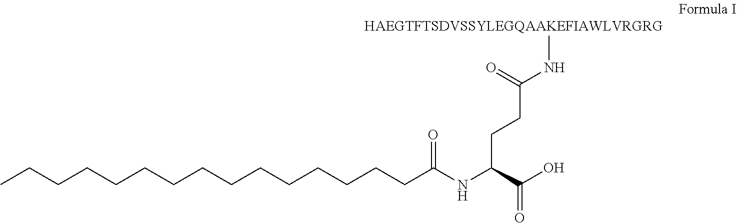 [SMILES: CCCCCCCCCCCCCCCC(=O)N[C@@H](CCC(=O)NC)C(=O)O]

In some embodiments, the long-acting GLP-1 receptor agonist is liraglutide that is co-formulated with insulin or an insulin analog. In some embodiments, provided is a long-acting GLP-1 receptor agonist of Formula II (SEQ ID NO:4), as described in U.S. Pat. No. 7,235,627:

Formula II
7 8 9 10 11 12 13 14 15 16 17
Xaa-Xaa-Glu-Gly-Thr-Phe-Thr-Ser-Asp-Val-Ser-
18 19 20 21 22 23 24 25 26 27 28
Xaa-Xaa-Xaa-Xaa-Xaa-Xaa-Xaa-Xaa-Xaa-Xaa-Phe-
29 30 31 32 33 34 35 36 37 38 39
Ile-Xaa-Xaa-Xaa-Xaa-Xaa-Xaa-Xaa-Xaa-Xaa-Xaa

wherein

Xaa at position 7 is His, a modified amino acid, or is deleted

Xaa at position 8 is Ala, Gly, Ser, Thr, Leu, Ile, Val, Glu, or Asp,

Xaa at position 18 is Ser, Ala, Gly, Thr, Leu, Ile, Val, Glu, Asp, or Lys,

Xaa at position 19 is Tyr, Phe, Trp, Glu, Asp, or Lys,

Xaa at position 20 is Leu, Ala, Gly, Ser, Thr, Leu, Ile, Val, Glu, Asp, or Lys,

Xaa at position 21 is Glu, Asp, or Lys,

Xaa at position 22 is Gly, Ala, Ser, Thr, Leu, Ile, Val, Glu, Asp, or Lys,

Xaa at position 23 is Gln, Asn, Arg, Glu, Asp, or Lys,

Xaa at position 24 is Ala, Gly, Ser, Thr, Leu, Ile, Val, Arg, Glu, Asp, or Lys,

Xaa at position 25 is Ala, Gly, Ser, Thr, Leu, Ile, Val, Glu, Asp, or Lys,

Xaa at position 26 is Lys, Arg, Gln, Glu, Asp, or His,

Xaa at position 27 is Glu, Asp, or Lys,

Xaa at position 30 is Ala, Gly, Ser, Thr, Leu, Ile, Val, Glu, Asp, or Lys,

Xaa at position 31 is Trp, Phe, Tyr, Glu, Asp, or Lys,

Xaa at position 32 is Leu, Gly, Ala, Ser, Thr, Ile, Val, Glu, Asp, or Lys,

Xaa at position 33 is Val, Gly, Ala, Ser, Thr, Leu, Ile, Glu, Asp, or Lys,

Xaa at position 34 is Lys, Arg, Glu, Asp, or His,

Xaa at position 35 is Gly, Ala, Ser, Thr, Leu, Ile, Val, Glu, Asp, or Lys,

Xaa at position 36 is Arg, Lys, Glu, Asp, or His,

Xaa at position 37 is Gly, Ala, Ser, Thr, Leu, Ile, Val, Glu, Asp, or Lys or is deleted

Xaa at position 38 is Arg, Lys, Glu, Asp, or His, or is deleted,

Xaa at position 39 is Arg or is deleted, or
(a) a C-1-6-ester thereof, (b) amide, C-1-6-alkylamide, or C-1-6-dialkylamide thereof and/or (c) a pharmaceutically acceptable salt thereof, provided that
   (i) when the amino acid at position 37 or 38 is deleted, then each amino acid downstream of the amino acid is also deleted,
   (ii) the long-acting GLP-1 receptor agonist contains only one Lys and the Lys is not the N-terminal or C-terminal amino acid of the derivative,
   (iii) a lipophilic substituent of from 12 to 25 carbons is attached, optionally via a spacer, to the ε-amino group of the Lys, and
the total number of different amino acids between the long-acting GLP-1 receptor agonist and the corresponding native form of GLP-1 does not exceed five.

In some embodiments, provided is the long-acting GLP-1 receptor agonist of Formula II:

wherein
Xaa at position 7 is His,
Xaa at position 8 is Ala,
Xaa at position 26 is Arg, Gln, Glu, Asp, or His, and
the total number of different amino acids between the long-acting GLP-1 receptor agonist and the corresponding native form of GLP-1 does not exceed three.
In some embodiments, Xaa at position 34 is Lys, Xaa at position 37 is Gly or is deleted, Xaa at position 38 is Arg or is deleted, and Xaa at position 39 is deleted.
In some embodiments, the total number of different amino acids between the long-acting GLP-1 receptor agonist and the corresponding native form of GLP-1 does not exceed two.
In some embodiments, the total number of different amino acids between the long-acting GLP-1 receptor agonist and the corresponding native form of GLP-1 is one.
In some embodiments, Xaa at position 34 is Arg, Glu, Asp, or His.
In some embodiments, Xaa at position 18 is Lys, Xaa at position 37 is Gly or is deleted, Xaa at position 38 is Arg or is deleted, Xaa at position 39 is deleted and each of the other Xaa is the amino acid in the native form of GLP-1 (7-36), (7-37) or (7-38).
In some embodiments, Xaa at position 23 is Lys, Xaa at position 37 is Gly or is deleted, Xaa at position 38 is Arg or is deleted, Xaa at position 39 is deleted and each of the other Xaa is the amino acid in the native form of GLP-1 (7-36), (7-37) or (7-38).
In some embodiments, Xaa at position 27 is Lys, Xaa at position 37 is Gly or is deleted, Xaa at position 38 is Arg or is deleted, Xaa at position 39 is deleted and each of the other Xaa is the amino acid in the native form of GLP-1 (7-36), (7-37) or (7-38).
In some embodiments, Xaa at position 36 is Lys, Xaa at position 37 is Gly, Xaa at position 38 is Arg or is deleted, Xaa at position 39 is deleted and each of the other Xaa is the amino acid in the native form of GLP-1(7-37) or (7-38).
In some embodiments, Xaa at position 38 is Lys, Xaa at position 39 is Arg and each of the other Xaa is the amino acid in the native form of GLP-1(7-39).
In some embodiments, Xaa at position 26 is Arg, Gln, Glu, Asp, or His.
In some embodiments, Xaa at position 34 is Arg, Glu, Asp, or His.
In some embodiments, Xaa at position 7 is His, and Xaa at position 8 is Ala.
In some embodiments, Xaa at position 7 is His, and Xaa at position 8 is Thr, Ser, Gly or Val.
In some embodiments, Xaa at position 7 is deleted.
In some embodiments, Xaa at position 8 is Ala.
In some embodiments, Xaa at position 8 is Thr, Ser, Gly or Val.
In some embodiments, Xaa at position 7 is a modified amino acid.
In some embodiments, Xaa at position 8 is Ala.
In some embodiments, Xaa at position 8 is Thr, Ser, Gly or Val.
In some embodiments, Xaa at position 18, 23 or 27 is Lys, Xaa at position 37 is Gly or is deleted, Xaa at position 38 is Arg or is deleted, and Xaa at position 39 is deleted.
In some embodiments, Xaa at position 36 is Lys, Xaa at position 37 is Gly, Xaa at position 38 is Arg, and Xaa at position 39 is deleted.
In some embodiments, Xaa at position 38 is Lys, Xaa at position 37 is Gly, and Xaa at position 39 is Arg.
In some embodiments, Xaa at position 34 is Lys, Xaa at position 37 is Gly or is deleted, Xaa at position 38 is Arg or is deleted, and Xaa at position 39 is deleted.
In some embodiments, Xaa at position 7 is His, and Xaa at position 8 is Ala.
In some embodiments, Xaa at position 7 is His, and Xaa at position 8 is Thr, Ser, Gly or Val.
In some embodiments, Xaa at position 7 is deleted.
In some embodiments, Xaa at position 8 is Ala.
In some embodiments, Xaa at position 8 is Thr, Ser, Gly or Val.
In some embodiments, Xaa at position 7 is a modified amino acid.
In some embodiments, Xaa at position 8 is Ala.
In some embodiments, Xaa at position 8 is Thr, Ser, Gly or Val.
In some embodiments, Xaa at position 26 is Lys and
Xaa at position 34 is Arg, Glu, Asp, or His, and
the total number of different amino acids between the long-acting GLP-1 receptor agonist and the corresponding native form of GLP-1(7-36), (7-37) or (7-38) does not exceed three.
In some embodiments, Xaa at position 7 is His, and Xaa at position 8 is Ala.
In some embodiments, Xaa at position 7 is His, and Xaa at position 8 is Thr, Ser, Gly or Val.
In some embodiments, Xaa at position 7 is a modified amino acid.
In some embodiments, Xaa at position 8 is Ala.
In some embodiments, Xaa at position 8 is Thr, Ser, Gly or Val.
In some embodiments, Xaa at position 7 is deleted.
In some embodiments, Xaa at position 8 is Ala.
In some embodiments, Xaa at position 8 is Thr, Ser, Gly or Val.

In some embodiments, provided is a long-acting GLP-1 receptor agonist of Formula III (SEQ ID NO:5), as described in U.S. Pat. No. 6,268,343:

wherein

Formula III
7 8 9 10 11 12 13 14 15 16 17
His-Ala-Glu-Gly-Thr-Phe-Thr-Ser-Asp-Val-Ser-
18 19 20 21 22 23 24 25 26 27 28
Ser-Tyr-Leu-Glu-Gly-Gln-Ala-Ala-Lys-Glu-Phe-
29 30 31 32 33 34 35 36 37
Ile-Ala-Trp-Leu-Val-Arg-Gly-Arg-Gly

(a) the ε-amino group of Lys at position 26 is substituted with a lipophilic substituent, optionally via a spacer,
(b) the lipophilic substituent is (i) CH₃(CH₂)ₙCO— wherein n is 6, 8, 10, 12, 14, 16, 18, 20 or 22, (ii) HOOC(CH₂)ₘCO— wherein m is 10, 12, 14, 16, 18, 20 or 22, or (iii) lithochoyl, and
(c) the spacer is (i) an unbranched alkane α,ω-dicarboxylic acid group having from 1 to 7 methylene groups, (ii) an amino acid residue except Cys, or (iii) γ-aminobutanoyl.

In some embodiments, provided is the long-acting GLP-1 receptor agonist of Formula III, wherein the lipophilic substituent is linked to the ε-amino group of Lys via a spacer. In some embodiments, the spacer is γ-glutamyl. In some embodiments, the spacer is β-asparagyl. In some embodiments, the spacer is glycyl. In some embodiments, the spacer is γ-aminobutanoyl. In some embodiments, the spacer is β-alanyl.

In some embodiments, the long-acting GLP-1 receptor agonist is Lys²⁶(N^{ε}-tetradecanoyl), Arg³⁴-GLP-1(7-37). In some embodiments, the long-acting GLP-1 receptor agonist is Lys²⁶(N^{ε}-(ω-carboxynonadecanoyl)), Arg³⁴-GLP-1(7-37). In some embodiments, the long-acting GLP-1 receptor agonist is Lys²⁶(N^{ε}-(ω-carboxyheptadecanoyl)), Arg³⁴-GLP-1(7-37). In some embodiments, the long-acting GLP-1 receptor agonist is Lys²⁶(N^{ε}-(ω-carboxyundecanoyl)), Arg³⁴-GLP-1(7-37). In some embodiments, the long-acting GLP-1 receptor agonist is Lys²⁶(N^{ε}-(ω-carboxypentadecanoyl)), Arg³⁴-GLP-1(7-37). In some embodiments, the long-acting GLP-1 receptor agonist is Lys²⁶(N^{ε}-lithochoyl),Arg³⁴-GLP-1(7-37). In some embodiments, the long-acting GLP-1 receptor agonist is Lys²⁶(N^{ε}-(γ-glutamyl(N^{α}-hexadecanoyl))), Arg³⁴-GLP-1(7-37). In some embodiments, the long-acting GLP-1 receptor agonist is Lys²⁶(N^{ε}-(γglutamyl(N^{α}tetradecanoyl))), Arg³⁴-GLP-1(7-37). In some embodiments, the long-acting GLP-1 receptor agonist is Lys²⁶(N^{ε}-(γglutamyl(N^{α}lithochoyl))), Arg³⁴-GLP-1(7-37). In some embodiments, the long-acting GLP-1 receptor agonist is Lys²⁶(N^{ε}-(γglutamyl(N^{α}octadecanoyl))), Arg³⁴-GLP-1(7-37). In some embodiments, the long-acting GLP-1 receptor agonist is Lys²⁶(N^{c}-decanoyl), Arg³⁴-GLP-1(7-37). In some embodiments, the long-acting GLP-1 receptor agonist is Lys²⁶(N-hexadecanoyl), Arg³⁴-GLP-1(7-37). In some embodiments, the long-acting GLP-1 receptor agonist is Lys²⁶(N-octanoyl), Arg³⁴-GLP-1(7-37). In some embodiments, the long-acting GLP-1 receptor agonist is Lys²⁶(N^{ε}-dodecanoyl), Arg³⁴-GLP-1(7-37). In some embodiments, the long-acting GLP-1 receptor agonist is Lys²⁶(N^{ε}(N⁶⁸(γaminobutyroyl-(N^{γ}-hexadecanoyl))), Arg³⁴-GLP-1(7-37). In some embodiments, the long-acting GLP-1 receptor agonist is Lys²⁶(N^{ε}-(γ-D-glutamyl(N^{α}hexadecanoyl))), Arg³⁴-GLP-1(7-37). In some embodiments, the long-acting GLP-1 receptor agonist is Lys²⁶(N^{ε}-(γglutamyl(N^{α}-dodecanoyl))), Arg³⁴-GLP-1(7-37). In some embodiments, the GLP-1 derivative is Lys²⁶(N^{ε}-(βalanyl(N^{α}-hexadecanoyl))), Arg³⁴-GLP-1(7-37). In some embodiments, the long-acting GLP-1 receptor agonist is Lys²⁶(N^{ε}-(α-glutamyl(N^{α}-hexadecanoyl))), Arg³⁴-GLP-1(7-37). In some embodiments, the long-acting GLP-1 receptor agonist is Lys²⁶(N^{ε}-(γ-glutamyl(N^{α}-decanoyl))), Arg³⁴-GLP-1(7-37).

Semaglutide (Ozempic®, developed and marketed by Novo Nordisk) is administered via weekly injection for treatment of type-2 diabetes. In some embodiments, the long-acting GLP-1 receptor agonist is semaglutide. In some embodiments, the long-acting GLP-1 receptor agonist is semaglutide that is co-formulated with insulin or an insulin analog. The structure of semaglutide is based on liraglutide, with two further modifications: Gly in position 8 is replaced by Aib. The spacer and lipophilic substituent of semaglutide join to form a N6-[N-(17-carboxy-1-oxoheptadecyl-L-c-glutamyl[2-(2-aminoethoxy)ethoxy]acetyl[2-(2-aminoethoxy)ethoxy]acetyl] residue.

In some embodiments, provided is the long-acting GLP-1 receptor agonist, semaglutide, of Formula IV (SEQ ID NO:6), as described in U.S. Pat. No. 8,129,343:

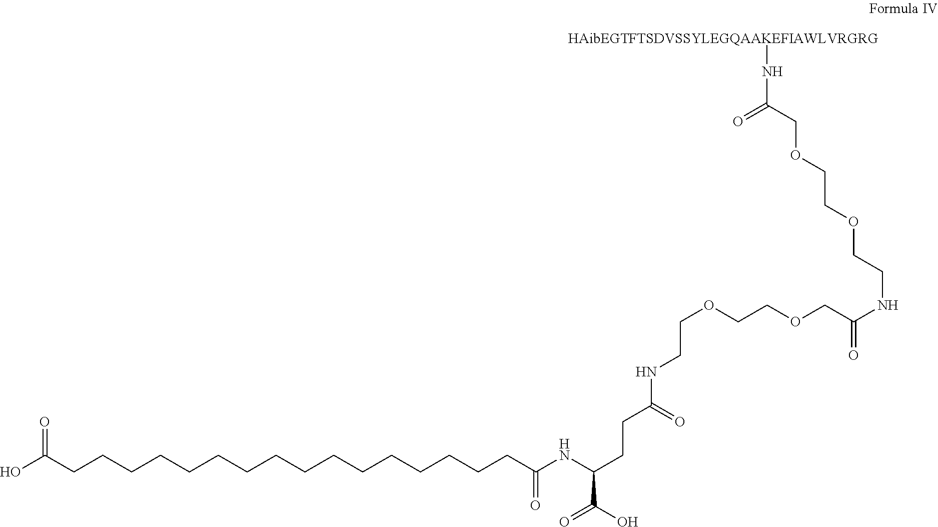

In some embodiments, provided is a long-acting GLP-1 receptor agonist of Formula V (SEQ ID NO:7), as described in U.S. Pat. Nos. 8,129,343 and 8,536,122:

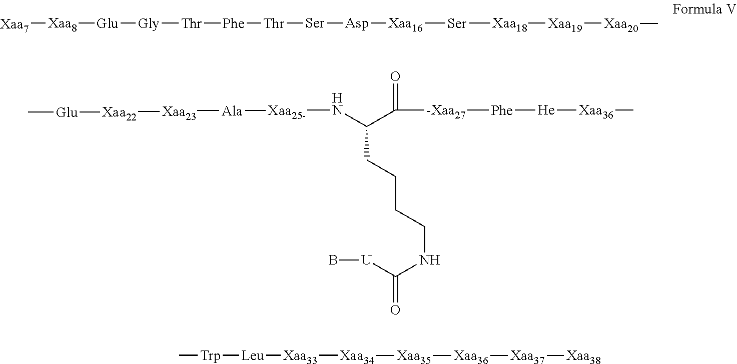

wherein

Xaa₇ is L-histidine, D-histidine, desamino-histidine, 2-amino-histidine, β-hydroxy-histidine, homohistidine, N^{α}-acetyl-histidine, a-fluoromethyl-histidine, a-methyl-histidine, 3-pyridylalanine, 2-pyridylalanine, or 4-pyridylalanine;

Xaa₈ is Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;

Xaa₁₆ is Val or Leu;

Xaa₁₈ is Ser, Lys, or Arg;

Xaa₁₉ is Tyr or Gin;

Xaa₂₀ is Leu or Met;

Xaa₂₂ is Gly, Glu, or Aib;

Xaa₂₃ is Gin, Glu, Lys, or Arg;

Xaa₂₅ is Ala or Val;

Xaa₂₇ is Glu or Leu;

Xaa₃₀ is Ala, Glu, or Arg;

Xaa₃₃ is Val or Lys;

Xaa₃₄ is Lys, Glu, Asn, or Arg;

Xaa₃₅ is Gly or Aib;

Xaa₃₆ is Arg, Gly, Lys, or is absent;

Xaa₃₇ is Gly, Ala, Glu, Pro, Lys, or is absent;

Xaa₃₈ is Lys, Ser, amide, or is absent; and

where U is a spacer selected from

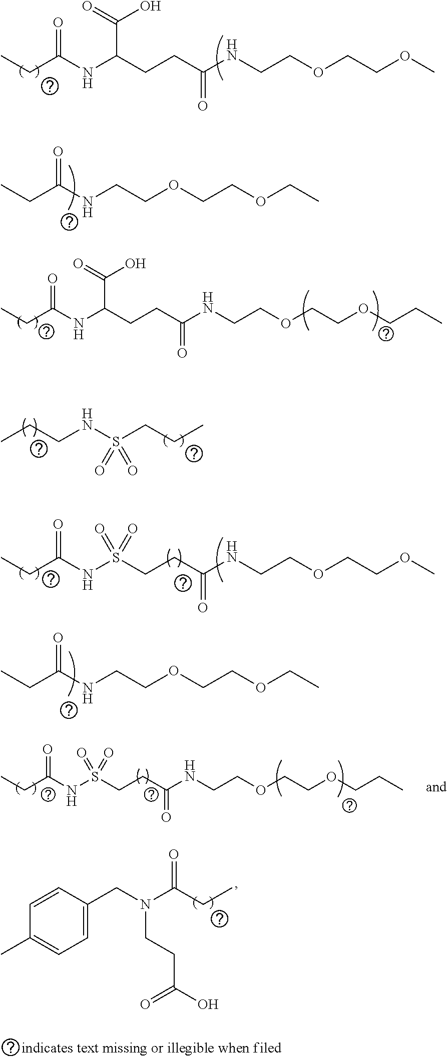

where n is 12, 13, 14, 15, 16, 17, or 18,

l is 12, 13, 14, 15, 16, 17, or 18,

m is 0, 1, 2, 3, 4, 5, or 6,

s is 0, 1, 2, or 3,

p is 3, 4, 5, 6, 7, 8, 9, 10, 11, 12, 13, 14, 15, 16, 17, 18, 19, 20, 21, 22, or 23; and

where B is an acidic group selected from

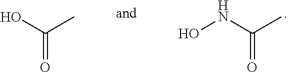

In some embodiments, provided is the long-acting GLP-1 receptor agonist of Formula V, wherein

Xaa₇ is His or desamino-histidine;

Xaa₈ is Gly, Val, Leu, Ile, Lys or Aib;

Xaa₁₆ is Val;

Xaa₁₈ is Ser;

Xaa₁₉ is Tyr;

Xaa₂₀ is Leu;

Xaa₂₂ is Gly, Glu or Aib;

Xaa₂₃ is Gln or Glu;

Xaa₂₅ is Ala;

Xaa₂₇ is Glu;

Xaa₃₀ is Ala or Glu;

Xaa₃₃ is Val;

Xaa₃₄ is Lys or Arg;

Xaa₃₅ is Gly or Aib;

Xaa₃₆ is Arg or Lys

Xaa₃₇ is Gly, amide or is absent; and

Xaa₃₈ is absent.

In some embodiments, provided is the long-acting GLP-1 receptor agonist of Formula V, wherein

Xaa₇ is His

Xaa₈ is Gly, or Aib;

Xaa₁₆ is Val;

Xaa₁₈ is Ser;

Xaa₁₉ is Tyr;

Xaa₂₀ is Leu;

Xaa₂₂ is Glu or Aib;

Xaa₂₃ is Gln;

Xaa₂₅ is Ala;

Xaa₂₇ is Glu;

Xaa₃₀ is Ala;

Xaa₃₃ is Val;

Xaa₃₄ is Lys or Arg;

Xaa₃₅ is Gly or Aib;

Xaa₃₆ is Arg

Xaa₃₇ is Gly and

Xaa₃₈ is absent.

In some embodiments, provided is the long-acting GLP-1 receptor agonist of Formula V, wherein said long-acting GLP-1 receptor agonist comprises Aib⁸ or Gly⁸ in position 8 of the GLP-1(7-37) sequence.

In some embodiments, provided is the long-acting GLP-1 receptor agonist of Formula V, wherein said long-acting GLP-1 receptor agonist comprises Aib⁸.

In some embodiments, provided is the long-acting GLP-1 receptor agonist of Formula V, wherein said long-acting GLP-1 receptor agonist comprises no more than six amino acid residues which have been exchanged, added or deleted as compared to GLP-1(7-37) set forth in the following sequence HAEGTFTSDVSSYLEGQAAKEFIAWLVKGRG (SEQ ID No: 8).

In some embodiments, provided is the long-acting GLP-1 receptor agonist of Formula V, wherein said long-acting GLP-1 receptor agonist comprises no more than 3 amino acid residues which have been exchanged, added or deleted as compared to GLP-1(7-37) (SEQ ID No: 8).

In some embodiments, provided is the long-acting GLP-1 receptor agonist of Formula V, wherein said long-acting GLP-1 receptor agonist comprises only one lysine residue.

In some embodiments, provided is the long-acting GLP-1 receptor agonist of Formula V, which is Aib⁸, Arg³⁴-GLP-1(7-37) or Aib^{8,22}, Arg³⁴-GLP-1(7-37).

In some embodiments, provided is the long-acting GLP-1 receptor agonist of Formula V, wherein U is a spacer selected from

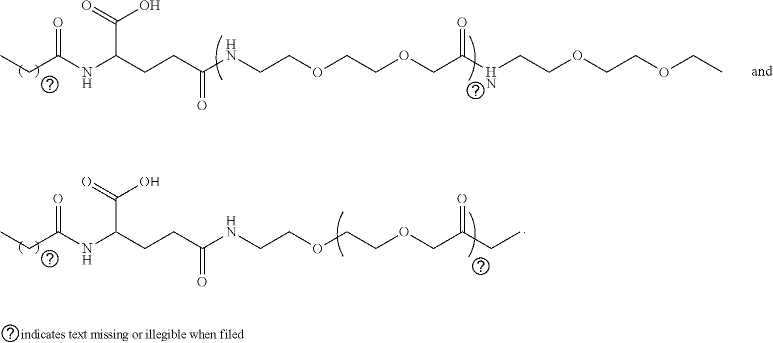

In some embodiments, B is

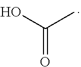

In some embodiments, provided is the long-acting GLP-1 receptor agonist having the following name: N-ε²⁶-[2-(2-[2-(2-[2-(2-[4-(17-Carboxyheptadecanoylamino)-4(S)-carboxybutyrylamino]ethoxy)ethoxy]acetylamino)ethoxy]ethoxy)acetyl][Aib8,Arg34]GLP-1-(7-37)peptide.

(iii) Conjugation of the GLP-1 Receptor Agonist to Albumin

Another half-life prolonging strategy is the fusion to recombinant albumin. Human serum albumin (HSA) has a molecular weight of about 67 kDa. The half-life of albumin in humans is about 19 days.

Albiglutide (Tanzeum®).

In some embodiments, the long-acting GLP-1 receptor agonist is albiglutide, developed by GlaxoSmithKline (GSK). Albiglutide includes two copies of GLP-1 fused as tandem repeat to the N-terminus of albumin. DPP-4-resistance is achieved by a single substitution, Ala for Gly, at the DPP-4 cleavage site. Albiglutide has a half-life of 6-8 days in humans.

Albiglutide has the following amino acid sequence (SEQ ID NO: 9):

HGEGTFTSDVSSYLEGQAAKEFIAWLVKGRHGEGTFTSDVSSYLEGQAAK
EFIAWLVKGRDAHKSEVAHREKDLGEENFKALVLIAFAQYLQQCPFEDHV
KLVNEVTEFAKTCVADESAENCDKSLHTLFGDKLCTVATLRETYGEMADC
CAKQEPERNECFLQHKDDNPNLPRLVRPEVDVMCTAFHDNEETFLKKYLY
EIARRHPYFYAPELLFFAKRYKAAFTECCQAADKAACLLPKLDELRDEGK
ASSAKQRLKCASLQKFGERAFKAWAVARLSQRFPKAEFAEVSKLVTDLTK
VHTECCHGDLLECADDRADLAKYICENQDSISSKLKECCEKPLLEKSHCI
AEVENDEMPADLPSLAADFVESKDVCKNYAEAKDVFLGMFLYEYARRHPD
YSVVLLLRLAKTYETTLEKCCAAADPHECYAKVFDEFKPLVEEPQNLIKQ
NCELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFSA
LEVDETYVPKEFNAETFTFHADICTLSEKERQIKKQTALVELVKHKPKAT
KEQLKAVMDDFAAFVEKCCKADDKETCFAEEGKKLVAASQAALGL

(iv) Conjugation of the GLP-1 Receptor Agonist to an Fc Region of Immunoglobulin G (IgG)

Fc Fusion:

Similar to albumin fusion, peptides can be linked to the constant region of immunoglobulin G (IgG), the Fc region. The Fc region of IgG has a half-life of about 22 days.

Dulaglutide

(Trulicity®, Eli Lilly) is a recombinant fusion protein, which consists of two GLP-1 peptides covalently linked by a small peptide [tetraglycyl-L-seryltetraglycyl-L-seryltetraglycyl-Lseryl-L-alanyl (SEQ ID NO: 12)] to a human IgG4-Fc heavy chain variant. The first 31 amino acids of dulaglutide are residues 3-37 of human GLP-1 with the following substitutions (relative to GLP-1 numbering): Ala8Gly, Gly22Glu, Arg36Gly to ensure protection from DPP-IV cleavage. The next 16 amino acids (GGGGGGGSGGGGSG (SEQ ID NO: 11)) are a linker sequence. The remaining 228 amino acids are a synthetic human Fc fragment (immunoglobulin G4). Two identical peptide chains form a dimer, linked by inter-monomer disulphide bonds between Cys55-55 and Cys58-58.

In some embodiments, provided is a long-acting GLP-1 receptor agonist, dulaglutide, having the following amino acid sequence (SEQ ID NO. 10):

| | |
|---|---|
| 0 | HGEGTFTSDVSSYLEEQAAKEFIAWLVKGGGGGGGSGGGGSGGGGSAESK |
| 50 | YGPPCPPCPAPEAAGGPSVFLFPPKPKDTLMISRTPEVTCVVVDVSQEDP |
| 100 | EVQFNWYVDGVEVHNAKTKPREEQFNSTYRVVSVLTVLHQDWLNGKEYKC |
| 150 | KVSNKGLPSSIEKTISKAKGQPREPQVYTLPPSQEEMTKNQVSLTCLVKG |
| 200 | FYPSDIAVEWESNGQPENNYKTTPPVLDSDGSFFLYSRLTVDKSRWQEGN |
| 250 | VFSCSVMHEALHNHYTQKSLSLSLG |

In some embodiments, the nauseogenic compound is a long-acting GLP-1 receptor agonist selected from any of the compounds of Formula I, Formula II, Formula III, Formula IV, and Formula V. In some embodiments, the nauseogenic compound is a long-acting GLP-1 receptor agonist of Formula I. In some embodiments, the nauseogenic compound is a long-acting GLP-1 receptor agonist of Formula II. In some embodiments, the nauseogenic compound is a long-acting GLP-1 receptor agonist of Formula III. In some embodiments, the nauseogenic compound is a long-acting GLP-1 receptor agonist of Formula IV. In some embodiments, the nauseogenic compound is a long-acting GLP-1 receptor agonist of Formula V.

In some embodiments, the nauseogenic compound is a long-acting GLP-1 receptor agonist selected from any of the compounds of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10. In some embodiments, the nauseogenic compound is a long-acting GLP-1 receptor agonist of SEQ ID NO. 1. In some embodiments, the nauseogenic compound is a long-acting GLP-1 receptor agonist of SEQ ID NO. 2. In some embodiments, the nauseogenic compound is a long-acting GLP-1 receptor agonist of SEQ ID NO. 3. In some embodiments, the nauseogenic compound is a long-acting GLP-1 receptor agonist of SEQ ID NO. 4. In some embodiments, the nauseogenic compound is a long-acting GLP-1 receptor agonist of SEQ ID NO. 5. In some embodiments, the nauseogenic compound is a long-acting GLP-1 receptor agonist of SEQ ID NO. 6. In some embodiments, the nauseogenic compound is a long-acting GLP-1 receptor agonist of SEQ ID NO. 7. In some embodiments, the nauseogenic compound is a long-acting GLP-1 receptor agonist of SEQ ID NO. 8. In some embodiments, the nauseogenic compound is a long-acting GLP-1 receptor agonist of SEQ ID NO. 9. In some embodiments, the nauseogenic compound is a long-acting GLP-1 receptor agonist of SEQ ID NO. 10.

### Area Postrema and Nauseogenic Peptides

The area postrema was identified in the early 1950's as the locus of the chemoreceptor zone responsible for triggering vomiting. The area postrema and adjacent structures within the dorsovagal complex, including the nucleus of the tractus solitaries (NTS) are rich in receptors for peptide hormones, and for gut peptides in particular. Peptide pharmacologies identified at area postrema neurons include those listed below. Some of the peptides below sensed at area postrema have been reported to inhibit food intake and potentially induce nausea. In certain embodiments, the nauseogenic peptide is selected from the group consisting of adrenomedullin, amylin, angiotensin II, atrial natriuretic peptide, cholecystokinin, chorionic gonadotropin leuteinizing hormone, corticotrophin releasing factor, endothelins, gastrin, ghrelin, glucagon, glucagon-like peptide 1 (GLP-1), insulin, insulin-like growth factor, leptin, leu-enkephalin, melanocortins, neurotensin, oxytocin, parathyroid hormones (e.g., PTH, PTHrP), pituitary adenylate cyclase activating peptide (PACAP), prolactin, prolactin releasing peptide, somatostatin, tachykinins (e.g., substance P), thyrotropin releasing hormone, vasoactive intestinal peptide (VIP), vasopressin, neuropeptide Y (NPY), pancreatic polypeptide (PP) and peptide YY (PYY), an agonist thereof, and an agonist of the receptor thereof. In some embodiments, the nauseogenic peptide is not insulin.

### Drug Particles, Suspension Vehicle, and Administration Via Drug Delivery Devices

In one aspect, the present invention provides formulations of drug particles suspended in a suspension vehicle for dispersion from a drug delivery device. The suspension vehicle provides a stable environment in which the drug particle formulation is dispersed. Certain features of the drug particle and suspension vehicle are described in greater detail below.

### Drug Particles

The particle formulation typically comprises a drug (i.e., the nauseogenic compound) and includes one or more stabilizing component (also referred to herein as “excipients”). Examples of stabilizing components include, but are not limited to, carbohydrates, antioxidants, amino acids, buffers, inorganic compounds, and surfactants.

In any of the embodiments, the particle formulation may comprise about 50 wt % to about 90 wt % drug, about 50 wt % to about 85 wt % drug, about 55 wt % to about 90 wt % drug, about 60 wt % to about 90 wt % drug, about 65 wt % to about 85 wt % drug, about 65 wt % to about 90 wt % drug, about 70 wt % to about 90 wt % drug, about 70 wt % to about 85 wt % drug, about 70 wt % to about 80 wt % drug, or about 70 wt % to about 75 wt % drug.

In any of the embodiments, a particle formulation comprises a drug, as described above, and one or more stabilizer. The stabilizers may be, for example, carbohydrate, antioxidant, amino acid, buffer, inorganic compound, or surfactant. The amounts of stabilizers in the particle formulation can be determined experimentally based on the activities of the stabilizers and the desired characteristics of the formulation, in view of the teachings of the present specification.

Examples of carbohydrates that may be included in the particle formulation include, but are not limited to, monosaccharides (e.g., fructose, maltose, galactose, glucose, D-mannose, and sorbose), disaccharides (e.g., lactose, sucrose, trehalose, and cellobiose), polysaccharides (e.g., raffinose, melezitose, maltodextrins, dextrans, and starches), and alditols (acyclic polyols; e.g., mannitol, xylitol, maltitol, lactitol, xylitol sorbitol, pyranosyl sorbitol, and myoinsitol). Suitable carbohydrates include disaccharides and/or non-reducing sugars, such as sucrose, trehalose, and raffinose.

Examples of antioxidants that may be included in the particle formulation include, but are not limited to, methionine, ascorbic acid, sodium thiosulfate, catalase, platinum, ethylenediaminetetraacetic acid (EDTA), citric acid, cysteine, thioglycerol, thioglycolic acid, thiosorbitol, butylated hydroxanisol, butylated hydroxyltoluene, and propyl gallate. Further, amino acids that readily oxidize can be used as antioxidants, for example, cysteine, methionine, and tryptophan.

Examples of amino acids that may be included in the particle formulation include, but are not limited to, arginine, methionine, glycine, histidine, alanine, L-leucine, glutamic acid, iso-leucine, L-threonine, 2-phenylamine, valine, norvaline, proline, phenylalanine, tryptophan, serine, asparagines, cysteine, tyrosine, lysine, and norleucine. Suitable amino acids include those that readily oxidize, e.g., cysteine, methionine, and tryptophan.

Examples of buffers that may be included in the particle formulation include, but are not limited to, citrate, histidine, succinate, phosphate, maleate, tris, acetate, carbohydrate, and gly-gly. Suitable buffers include citrate, histidine, succinate, and tris.

Examples of inorganic compounds that may be included in the particle formulation include, but are not limited to, NaCl, Na₂SO₄, NaHCO₃, KCl, KH₂PO₄, CaCl₂, and MgCl₂.

In addition, the particle formulation may include other stabilizers/excipients, such as surfactants and salts. Examples of surfactants include, but are not limited to, Polysorbate 20, Polysorbate 80, PLURONIC® (BASF Corporation, Mount Olive, N.J.) F68, and sodium dodecyl sulfate (SDS). Examples of salts include, but are not limited to, sodium chloride, calcium chloride, and magnesium chloride.

Drug particle formulations of the invention are preferably chemically and physically stable for at least 1 month, preferably at least 3 months, more preferably at least 6 months, more preferably at least 12 months at delivery temperature. The delivery temperature is typically normal human body temperature, for example, about 37° C., or slightly higher, for example, about 40° C. Further, drug particle formulations of the present invention are preferably chemically and physically stable for at least 3 months, preferably at least 6 months, more preferably at least 12 months, at storage temperature. Examples of storage temperatures include refrigeration temperature, for example, about 5° C.; or room temperature, for example, about 25° C.

A drug particle formulation may be considered chemically stable if less than about 25%; preferably less than about 20%, more preferably less than about 15%, more preferably less than about 10%, and more preferably less than about 5% breakdown products of the drug particles are formed after about 3 months, preferably after about 6 months, preferably after about 12 months at delivery temperature of about 37° C. and after about 6 months, after about 12 months, and preferably after about 24 months at storage temperature of about 5° C. or about 25° C.

A drug particle formulation may be considered physically stable if less than about 10%, preferably less than about 5%, more preferably less than about 3%, more preferably less than 1% aggregates of the drug are formed after about 3 months, preferably after about 6 months, at delivery temperature and about 6 months, preferably about 12 months, at storage temperature.

The particles are typically sized such that they can be delivered via an implantable osmotic delivery device. Uniform shape and size of the particles typically helps to provide a consistent and uniform rate of release from such a delivery device; however, a particle preparation having a non-normal particle size distribution profile may also be used. For example, in a typical implantable osmotic delivery device having a delivery orifice, the size of the particles is less than about 30%, more preferably is less than about 20%, more preferably is less than about than 10%, of the diameter of the delivery orifice. In an embodiment of the particle formulation for use with an osmotic delivery system, wherein the delivery orifice diameter of the implant is about 0.5 mm, particle sizes may be, for example, less than about 150 microns to about 50 microns. In an embodiment of the particle formulation for use with an osmotic delivery system, wherein the delivery orifice diameter of the implant is about 0.1 mm, particle sizes may be, for example, less than about 30 microns to about 10 microns. In one embodiment, the orifice is about 0.25 mm (250 microns) and the particle size is about 2 microns to about 5 microns.

Those of ordinary skill in the art will appreciate that a population of particles follow principles of particle size distribution. Widely used, art-recognized methods of describing particle size distributions include, for example, average diameters and D values, such as the D₅₀ value, which is commonly used to represent the mean diameter of the range of the particle sizes of a given sample.

In some embodiments, particles of a particle formulation have average diameters of about 1 micron to about 150 microns, e.g., less than 150 microns in diameter, less than 100 microns in diameter, less than 50 microns in diameter, less than 30 microns in diameter, less than 10 microns in diameter, less than 5 microns in diameter, and less than about 2 microns in diameter. In some embodiments, particles have average diameters of about 1 micron and about 50 microns. In some embodiments, particles of a particle formulation have average diameters of less than 1 micron.

Particles of a particle formulation comprising a nauseogenic compound may have average diameters, e.g., of about 0.3 microns to about 150 microns. Particles of a particle formulation comprising an nauseogenic compound have average diameters of about 2 microns to about 150 microns, e.g., less than 150 microns in average diameter, less than 100 microns in average diameter, less than 50 microns in average diameter, less than 30 microns in average diameter, less than 10 microns in average diameter, less than 5 microns in average diameter, and about 2 microns in average diameter. In some embodiments, particles have average diameters of about 0.3 microns and 50 microns, for example, about 2 microns and about 50 microns. In some embodiments, the particles have an average diameter between 0.3 microns and 50 microns, for example, between about 2 microns and about 50 microns, where each particle is less than about 50 microns in diameter.

Typically, the particles of the particle formulations, when incorporated in a suspension vehicle, do not settle in less than about 3 months, preferably do not settle in less than about 6 months, more preferably do not settle in less than about 12 months, more preferably do not settle in less than about 24 months at delivery temperature, and most preferably do not settle in less than about 36 months at delivery temperature of about 37° C. The suspension vehicles typically have a viscosity of between about 5,000 to about 30,000 poise, preferably between about 8,000 to about 25,000 poise, more preferably between about 10,000 to about 20,000 poise. In one embodiment, the suspension vehicle has a viscosity of about 15,000 poise, plus or minus about 3,000 poise. Generally speaking, smaller particles tend to have a lower settling rate in viscous suspension vehicles than larger particles. Accordingly, micron- to nano-sized particles are typically desirable. In viscous suspension formulation, particles of about 2 microns to about 7 microns of the present invention will not settle for at least 20 years at room temperature based on simulation modeling studies. In an embodiment of the particle formulation of the present invention, for use in an implantable osmotic delivery device, comprises particles of sizes less than about 50 microns, more preferably less than about 10 microns, more preferably in a range from about 2 microns to about 7 microns.

In some embodiments, particles of the particle formulations have a specific density that is substantially similar (e.g., within 20%, 10%, 5%, 2% or 1%) to the specific density of the suspension vehicle to minimize separation (e.g., floating or settling) of the particles from the suspension vehicle.

In one embodiment, a drug particle formulation comprises a drug, as described above, one or more stabilizers, and optionally a buffer. The stabilizers may be, for example, carbohydrate, antioxidant, amino acid, buffer, inorganic compound, or surfactant.

Examples of carbohydrates that may be included in the particle formulation include, but are not limited to, monosaccharides (e.g., fructose, maltose, galactose, glucose, D-mannose, and sorbose), disaccharides (e.g., lactose, sucrose, trehalose, and cellobiose), polysaccharides (e.g., raffinose, melezitose, maltodextrins, dextrans, and starches), and alditols (acyclic polyols; e.g., mannitol, xylitol, maltitol, lactitol, xylitol sorbitol, pyranosyl sorbitol, and myoinsitol). Preferred carbohydrates include disaccharides and/or non-reducing sugars, such as sucrose, trehalose, and raffinose.

Examples of antioxidants that may be included in the particle formulation include, but are not limited to, methionine, ascorbic acid, sodium thiosulfate, catalase, platinum, ethylenediaminetetraacetic acid (EDTA), citric acid, cysteine, thioglycerol, thioglycolic acid, thiosorbitol, butylated hydroxanisol, butylated hydroxyltoluene, and propyl gallate. Further, amino acids that readily oxidize can be used as antioxidants, for example, cysteine, methionine, and tryptophan.

Examples of amino acids that may be included in the particle formulation include, but are not limited to, arginine, methionine, glycine, histidine, alanine, L-leucine, glutamic acid, iso-leucine, L-threonine, 2-phenylamine, valine, norvaline, praline, phenylalanine, tryptophan, serine, asparagines, cysteine, tyrosine, lysine, and norleucine.

Examples of buffers that may be included in the particle formulation include, but are not limited to, citrate, histidine, succinate, phosphate, maleate, tris, acetate, carbohydrate, and gly-gly.

Examples of inorganic compounds that may be included in the particle formulation include, but are not limited to, NaCl, Na₂SO₄, NaHCO₃, KCl, KH₂PO₄, CaCl₂, and MgCl₂.

In addition, the particle formulation may include other excipients, such as surfactants, and salts. Examples of surfactants include, but are not limited to, Polysorbate 20, Polysorbate 80, PLURONIC® (BASF Corporation, Mount Olive, N.J.) F68, and sodium dodecyl sulfate (SDS). Examples of salts include, but are not limited to, sodium chloride, calcium chloride, and magnesium chloride.

All components included in the particle formulation are typically acceptable for pharmaceutical use in subjects, patients, mammals, particularly, in humans.

In summary, a selected drug or combination of drugs is formulated into dried powders in solid state, which preserve maximum chemical and biological stability of the drug. The particle formulation offers long-term storage stability at high temperature, and therefore, allows delivery to a subject of stable and biologically effective drug for extended periods of time.

### Suspension Vehicle

In one aspect, the suspension vehicle provides a stable environment in which the drug particle formulation is dispersed. The drug particle formulations are chemically and physically stable (as described above) in the suspension vehicle. The suspension vehicle typically comprises one or more polymer and one or more solvent that form a solution of sufficient viscosity to uniformly suspend the particles comprising the drug. The suspension vehicle may comprise further components, including, but not limited to, surfactants, antioxidants, and/or other compounds soluble in the vehicle.

The viscosity of the suspension vehicle is typically sufficient to prevent the drug particle formulation from settling during storage and use in a method of delivery, for example, in an implantable, osmotic delivery device. The suspension vehicle is biodegradable in that the suspension vehicle disintegrates or breaks down over a period of time in response to a biological environment, while the drug particle is dissolved in the biological environment and the active pharmaceutical ingredient (i.e., the drug) in the particle is absorbed.

In embodiments, the suspension vehicle is a “single-phase” suspension vehicle, which is a solid, semisolid, or liquid homogeneous system that is physically and chemically uniform throughout.

The solvent in which the polymer is dissolved may affect characteristics of the suspension formulation, such as the behavior of drug particle formulation during storage. A solvent may be selected in combination with a polymer so that the resulting suspension vehicle exhibits phase separation upon contact with the aqueous environment. In some embodiments of the invention, the solvent may be selected in combination with the polymer so that the resulting suspension vehicle exhibits phase separation upon contact with the aqueous environment having less than approximately about 10% water.

The solvent may be an acceptable solvent that is not miscible with water. The solvent may also be selected so that the polymer is soluble in the solvent at high concentrations, such as at a polymer concentration of greater than about 30%. Examples of solvents useful in the practice of the present invention include, but are not limited to, lauryl alcohol, benzyl benzoate, benzyl alcohol, lauryl lactate, decanol (also called decyl alcohol), ethyl hexyl lactate, and long chain (C₈ to C₂₄) aliphatic alcohols, esters, or mixtures thereof. The solvent used in the suspension vehicle may be “dry,” in that it has a low moisture content. Preferred solvents for use in formulation of the suspension vehicle include lauryl lactate, lauryl alcohol, benzyl benzoate, and mixtures thereof.

Examples of polymers for formulation of the suspension vehicles of the present invention include, but are not limited to, a polyester (e.g., polylactic acid and polylacticpolyglycolic acid), a polymer comprising pyrrolidones (e.g., polyvinylpyrrolidone having a molecular weight ranging from approximately 2,000 to approximately 1,000,000), ester or ether of an unsaturated alcohol (e.g., vinyl acetate), polyoxyethylenepolyoxypropylene block copolymer, or mixtures thereof. Polyvinylpyrrolidone can be characterized by its K-value (e.g., K-17), which is a viscosity index. In one embodiment, the polymer is polyvinylpyrrolidone having a molecular weight of 2,000 to 1,000,000. In a preferred embodiment, the polymer is polyvinylpyrrolidone K-17 (typically having an approximate average molecular weight range of 7,900-10,800). The polymer used in the suspension vehicle may include one or more different polymers or may include different grades of a single polymer. The polymer used in the suspension vehicle may also be dry or have a low moisture content.

Generally speaking, a suspension vehicle for use in the present invention may vary in composition based on the desired performance characteristics. In one embodiment, the suspension vehicle may comprise about 40 wt % to about 80 wt % polymer(s) and about 20 wt % to about 60 wt % solvent(s). Preferred embodiments of a suspension vehicle include vehicles formed of polymer(s) and solvent(s) combined at the following ratios: about 25 wt % solvent and about 75 wt % polymer; about 50 wt % solvent and about 50 wt % polymer; about 75 wt % solvent and about 25 wt % polymer. Accordingly, in some embodiments, the suspension vehicle may comprise selected components and in other embodiments consist essentially of selected components.

The suspension vehicle is typically formulated to provide a viscosity that maintains a uniform dispersion of the particle formulation for a predetermined period of time. This helps facilitate making a suspension formulation tailored to provide controlled delivery of the drug contained in the drug particle formulation. The viscosity of the suspension vehicle may vary depending on the desired application, the size and type of the particle formulation, and the loading of the particle formulation in the suspension vehicle. The viscosity of the suspension vehicle may be varied by altering the type or relative amount of the solvent or polymer used.

The suspension vehicle may have a viscosity ranging from about 100 poise to about 1,000,000 poise, preferably from about 1,000 poise to about 100,000 poise. In preferred embodiments, the suspension vehicles typically have a viscosity, at 33° C., of between about 5,000 to about 30,000 poise, preferably between about 8,000 to about 25,000 poise, more preferably between about 10,000 to about 20,000 poise. In one embodiment, the suspension vehicle has a viscosity of about 15,000 poise, plus or minus about 3,000 poise, at 33° C. The viscosity may be measured at 33° C., at a shear rate of 10⁻⁴/sec, using a parallel plate rheometer.

The suspension vehicle may exhibit phase separation when contacted with the aqueous environment; however, typically the suspension vehicle exhibits substantially no phase separation as a function of temperature. For example, at a temperature ranging from approximately 0° C. to approximately 70° C. and upon temperature cycling, such as cycling from 4° C. to 37° C. to 4° C., the suspension vehicle typically exhibits no phase separation.

The suspension vehicle may be prepared by combining the polymer and the solvent under dry conditions, such as in a dry box. The polymer and solvent may be combined at an elevated temperature, such as from approximately 40° C. to approximately 70° C., and allowed to liquefy and form the single phase. The ingredients may be blended under vacuum to remove air bubbles produced from the dry ingredients. The ingredients may be combined using a conventional mixer, such as a dual helix blade or similar mixer, set at a speed of approximately 40 rpm. However, higher speeds may also be used to mix the ingredients. Once a liquid solution of the ingredients is achieved, the suspension vehicle may be cooled to room temperature. Differential scanning calorimetry (DSC) may be used to verify that the suspension vehicle is a single phase. Further, the components of the vehicle (e.g., the solvent and/or the polymer) may be treated to substantially reduce or substantially remove peroxides (e.g., by treatment with methionine; see, e.g., U.S., Patent Application Publication No. 2007-0027105).

The drug particle formulation is added to the suspension vehicle to form a suspension formulation. In some embodiments, the suspension formulation may comprise a drug particle formulation and a suspension vehicle and in other embodiments consist essentially of a drug particle formulation and a suspension vehicle.

The suspension formulation may be prepared by dispersing the particle formulation in the suspension vehicle. The suspension vehicle may be heated and the particle formulation added to the suspension vehicle under dry conditions. The ingredients may be mixed under vacuum at an elevated temperature, such as from about 40° C. to about 70° C. The ingredients may be mixed at a sufficient speed, such as from about 40 rpm to about 120 rpm, and for a sufficient amount of time, such as about 15 minutes, to achieve a uniform dispersion of the particle formulation in the suspension vehicle. The mixer may be a dual helix blade or other suitable mixer. The resulting mixture may be removed from the mixer, sealed in a dry container to prevent water from contaminating the suspension formulation, and allowed to cool to room temperature before further use, for example, loading into an implantable, drug delivery device, unit dose container, or multiple-dose container.

The suspension formulation typically has an overall moisture content of less than about 10 wt %, preferably less than about 5 wt %, and more preferably less than about 4 wt %.

In preferred embodiments, the suspension formulations of the present invention are substantially homogeneous and flowable to provide delivery of the drug particle formulation from the osmotic delivery device to the subject.

In summary, the components of the suspension vehicle provide biocompatibility. Components of the suspension vehicle offer suitable chemico-physical properties to form stable suspensions of drug particle formulations. These properties include, but are not limited to, the following: viscosity of the suspension; purity of the vehicle; residual moisture of the vehicle; density of the vehicle; compatibility with the dry powders; compatibility with implantable devices; molecular weight of the polymer; stability of the vehicle; and hydrophobicity and hydrophilicity of the vehicle. These properties can be manipulated and controlled, for example, by variation of the vehicle composition and manipulation of the ratio of components used in the suspension vehicle.

### Delivery Via Implantable Delivery Devices

The suspension formulations described herein may be used in an implantable delivery device, including any of those described herein. In some embodiments, suspension formulations described herein may be used in an implantable, osmotic delivery device to provide zero-order, continuous, controlled, and sustained delivery of a compound over an extended period of time, such as over weeks, months, or up to about one year or more. Such an implantable osmotic delivery device is typically capable of delivering the suspension formulation, comprising the drug, at a desired flow rate over a desired period of time. The suspension formulation may be loaded into the implantable, osmotic delivery device by conventional techniques.

The implantable, osmotic delivery device typically includes a reservoir having at least one orifice through which the suspension formulation is delivered. The suspension formulation may be stored within the reservoir. In a preferred embodiment, the implantable, drug delivery device is an osmotic delivery device, wherein delivery of the drug is osmotically driven. Some osmotic delivery devices and their component parts have been described, for example, the DUROS® delivery device or similar devices (see, e.g., U.S. Pat. Nos. 5,609,885; 5,728,396; 5,985,305; 5,997,527; 6,113,938; 6,132,420; 6,156,331; 6,217,906; 6,261,584; 6,270,787; 6,287,295; 6,375,978; 6,395,292; 6,508,808; 6,544,252; 6,635,268; 6,682,522; 6,923,800; 6,939,556; 6,976,981; 6,997,922; 7,014,636; 7,207,982; and 7,112,335; 7,163,688; U.S. Patent Publication Nos. 2005/0175701, 2007/0281024, 2008/0091176, and 2009/0202608).

The osmotic delivery device typically consists of a cylindrical reservoir which contains the osmotic engine, piston, and drug formulation. The reservoir is capped at one end by a controlled-rate, semi-permeable membrane and capped at the other end by a diffusion moderator through which suspension formulation, comprising the drug, is released from the drug reservoir. The piston separates the drug formulation from the osmotic engine and utilizes a seal to prevent the water in the osmotic engine compartment from entering the drug reservoir. The diffusion moderator is designed, in conjunction with the drug formulation, to prevent body fluid from entering the drug reservoir through the orifice.

The osmotic device releases a drug at a predetermined rate based on the principle of osmosis. Extracellular fluid enters the osmotic delivery device through a semi-permeable membrane directly into a salt engine that expands to drive the piston at a slow and even delivery rate. Movement of the piston forces the drug formulation to be released through the orifice or exit port at a predetermined sheer rate. In one embodiment of the present invention, the reservoir of the osmotic device is loaded with a suspension formulation wherein the device is capable of delivering the suspension formulation to a subject over an extended period of time (e.g., about 1, about 3, about 6, about 9, about 10, and about 12 months) at a pre-determined, therapeutically effective delivery rate.

The release rate of the drug from the osmotic delivery device typically provides a subject with a predetermined target dose of a drug, for example, a therapeutically effective daily dose delivered over the course of a day; that is, the release rate of the drug from the device, provides substantial steady-state delivery of the drug at a therapeutic concentration to the subject.

Typically, for an osmotic delivery device, the volume of a beneficial agent chamber comprising the beneficial agent formulation is between about 100 μl to about 1000 μl, more preferably between about 120 μl and about 500 μl, more preferably between about 150 μl and about 200 μl.

Typically, the osmotic delivery device is implanted within the subject, for example, subdermally or subcutaneously to provide subcutaneous drug delivery. The device(s) can be implanted subdermally or subcutaneously into either or both arms (e.g., in the inside, outside, or back of the upper arm) or the abdomen. Preferred locations in the abdominal area are under the abdominal skin in the area extending below the ribs and above the belt line. To provide a number of locations for implantation of one or more osmotic delivery device within the abdomen, the abdominal wall can be divided into 4 quadrants as follows: the upper right quadrant extending at least 2-3 centimeters below the right ribs, e.g., at least about 5-8 centimeters below the right ribs, and at least 2-3 centimeters to the right of the midline, e.g., at least about 5-8 centimeters to the right of the midline; the lower right quadrant extending at least 2-3 centimeters above the belt line, e.g., at least about 5-8 centimeters above the belt line, and at least 2-3 centimeters to the right of the midline, e.g., at least about 5-8 centimeters to the right of the midline; the upper left quadrant extending at least 2-3 centimeters below the left ribs, e.g., at least about 5-8 centimeters below the left ribs, and at least 2-3 centimeters to the left of the midline, e.g., at least about 5-8 centimeters to the left of the midline; and the lower left quadrant extending at least 2-3 centimeters above the belt line, e.g., at least about 5-8 centimeters above the belt line, and at least 2-3 centimeters to the left of the midline, e.g., at least about 5-8 centimeters to the left of the midline. This provides multiple available locations for implantation of one or more devices on one or more occasions. Implantation and removal of osmotic delivery devices are generally carried out by medical professionals using local anesthesia (e.g., lidocaine).

Termination of treatment by removal of an osmotic delivery device from a subject is straightforward, and provides the important advantage of immediate cessation of delivery of the drug to the subject.

Preferably, the osmotic delivery device has a fail-safe mechanism to prevent an inadvertent excess or bolus delivery of drug in a theoretical situation like the plugging or clogging of the outlet (diffusion moderator) through which the drug formulation is delivered. To prevent an inadvertent excess or bolus delivery of drug the osmotic delivery device is designed and constructed such that the pressure needed to partially or wholly dislodge or expel the diffusion moderator from the reservoir exceeds the pressure needed to partially or wholly dislodge or expel the semi-permeable membrane to the extent necessary to de-pressurize the reservoir. In such a scenario, pressure would build within the device until it would push the semi-permeable membrane at the other end outward, thereby releasing the osmotic pressure. The osmotic delivery device would then become static and no longer deliver the drug formulation provided that the piston is in a sealing relationship with the reservoir.

The suspension formulations may also be used in infusion pumps, for example, the ALZET® (DURECT Corporation, Cupertino, Calif.) osmotic pumps which are miniature, infusion pumps for the continuous dosing of laboratory animals (e.g., mice and rats).

### Delivery Via Non-Implantable Delivery Devices

The suspension formulations described herein may be used in an non-implantable delivery device, including any of those described herein. In some embodiments, the non-implantable delivery device is a miniaturized patch pump placed on the surface of the skin, such as e.g., JewelPUMP™ (Debiotech S.A.). Dosing of the JewelPUMP™ device is adjustable and programmable. As such, mean steady state concentration (Cₛₛ) in plasma of a nauseogenic compound can gradually be attained, via slow ramp-up of an increasing dosage, or via continuous administration of a fixed dose, in the subject over days, weeks or months. The JewelPUMP™ is based on a microelectromechanical system (MEMS) integrated and ultra-precise disposable pump-chip technology. The JewelPUMP™ is a miniaturized patch-pump with a disposable unit having payload for administration of compound. The disposable unit is filled once with compound and discarded after use, while the controller unit (including the electronics) can be used for 2 years with multiple disposable units. In some embodiments, the JewelPUMP™ is detachable, watertight for bathing and swimming, includes direct access bolus buttons and a discreet vibration & audio alarm on the patch-pump. In some embodiments, the JewelPUMP™ is remotely controlled.

### Uses

The above drugs and other drugs known to those of skill in the art are useful in methods of treatment for a “variety of conditions” including but not limited to the following: chronic pain, hemophilia and other blood disorders, endocrine disorders, metabolic disorders, non-alcoholic fatty liver disease (NAFLD), non-alcoholic steatohepatitis (NASH), Alzheimer's disease, cardiovascular diseases (e.g., heart failure, atherosclerosis, and acute coronary syndrome), rheumatologic disorders, diabetes (including type 1, type 2 diabetes mellitus, human immunodeficiency virus treatment-induced, latent autoimmune diabetes in adults, and steroid-induced), obesity, hypoglycemia unawareness, restrictive lung disease, chronic obstructive pulmonary disease, lipoatrophy, metabolic syndrome, leukemia, hepatitis, renal failure, infectious diseases (including bacterial infection, viral infection (e.g., infection by human immunodeficiency virus, hepatitis C virus, hepatitis B virus, yellow fever virus, West Nile virus, Dengue virus, Marburg virus, and Ebola virus), and parasitic infection), hereditary diseases (such as cerebrosidase deficiency and adenosine deaminase deficiency), hypertension, septic shock, autoimmune diseases (e.g., Grave's disease, systemic lupus erythematosus, multiple sclerosis, and rheumatoid arthritis), shock and wasting disorders, cystic fibrosis, lactose intolerance, Crohn's diseases, inflammatory bowel disease, gastrointestinal cancers (including colon cancer and rectal cancer, breast cancer, leukemia, lung cancer, bladder cancer, kidney cancer, non-Hodgkin lymphoma, pancreatic cancer, thyroid cancer, endometrial cancer, and other cancers). Further, some of the above agents are useful for the treatment of infectious diseases requiring chronic treatments including, but not limited to, tuberculosis, malaria, leishmaniasis, trypanosomiasis (sleeping sickness and Chagas disease), and parasitic worms.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1. Plasma Concentration Profiles

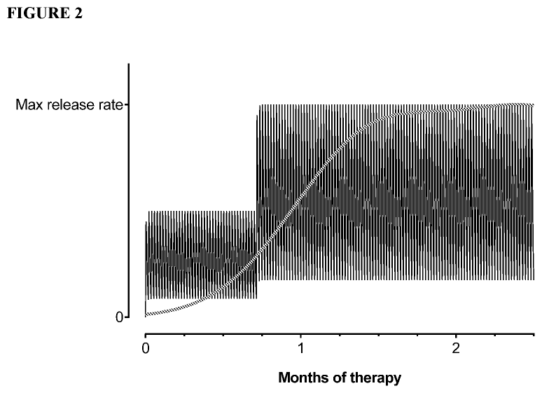

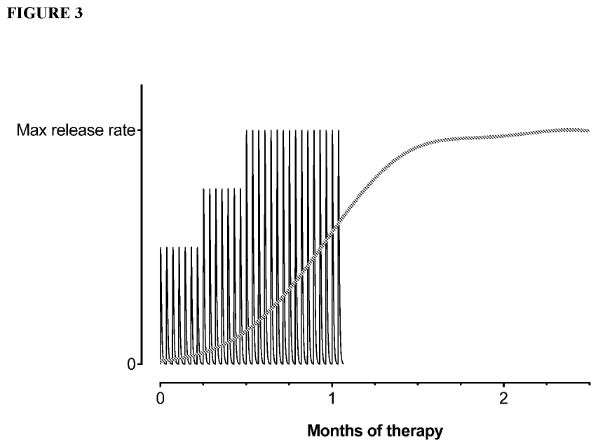

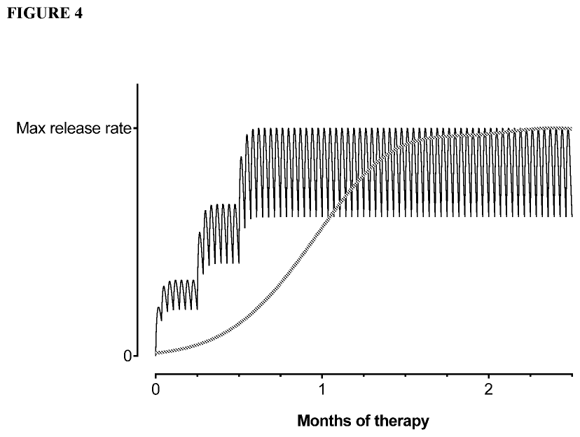

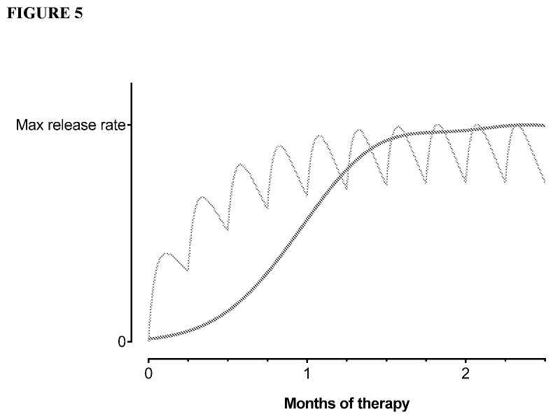

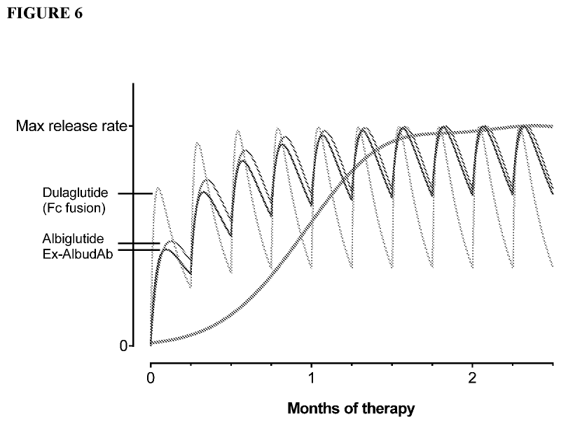

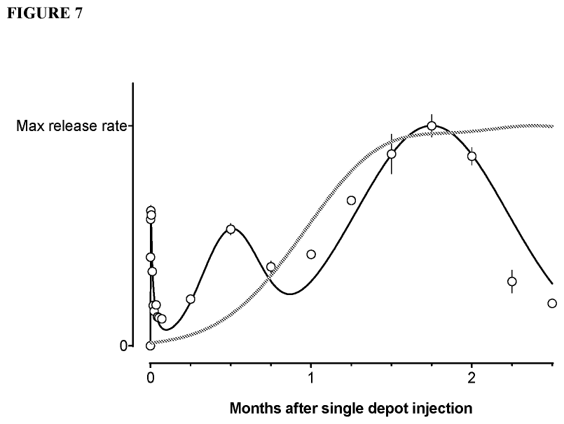

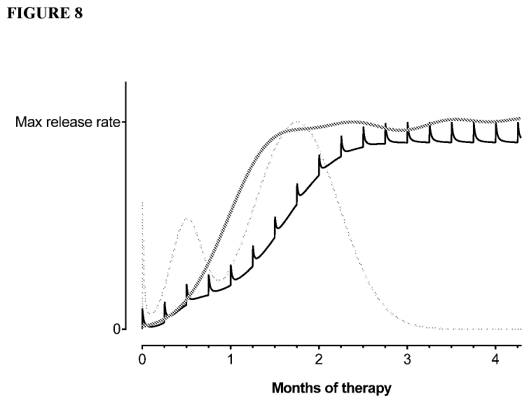

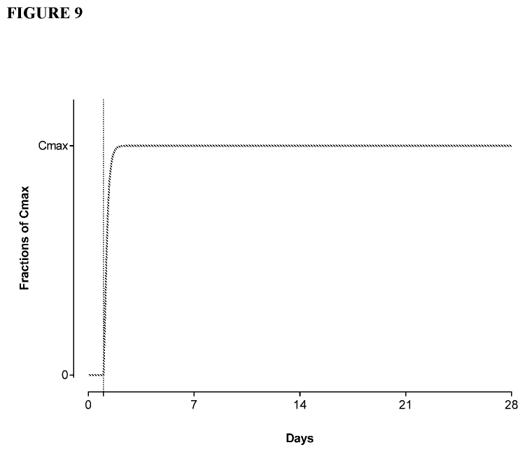

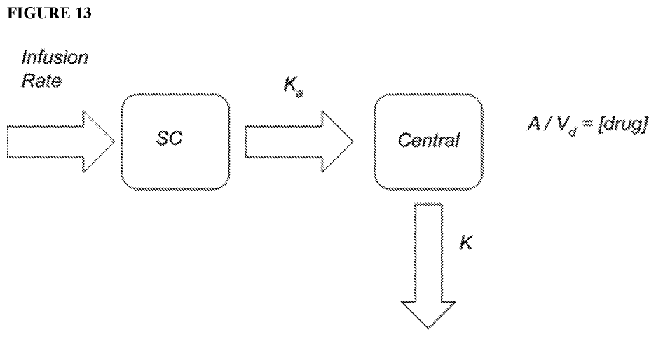

FIGS. 2-8 describe predicted plasma concentrations for different GLP-1 agonists dosed according to prescribers' information. Plasma concentrations are each expressed as a fraction of peak plasma concentration i.e., steady state concentration (Cₛₛ). The predictions are based upon published human pharmacokinetic data, with either raw plasma concentration data or data digitized from published figures for a single subcutaneous dose. The plasma concentrations were fitted to a model describing “absorption+single component decay” for each agent, as depicted in FIG. 13, based upon a rate constant for absorption from a subcutaneous depot into the plasma, and another rate constant for elimination from the plasma compartment. Data were fit using non-linear regression via an iterative least-squares method within Prism v7.0 (GraphPad Software Inc., San Diego, Calif.).

The derived plasma concentration profile for a single subcutaneous bolus was extended from the time of dosing until plasma concentrations were negligible. This profile was serially added to itself, staggered by a period determined by the indicated dose interval, and in a magnitude determined by the recommended dose increases. The numeric sum is shown as the black line in each plot.

An ideal plasma concentration profile is shown as a heavy orange line in each plot.

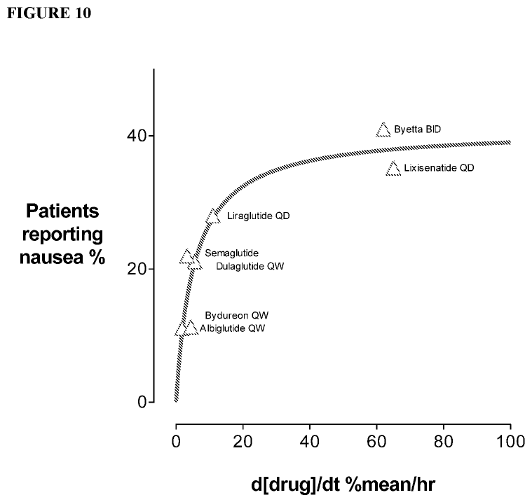

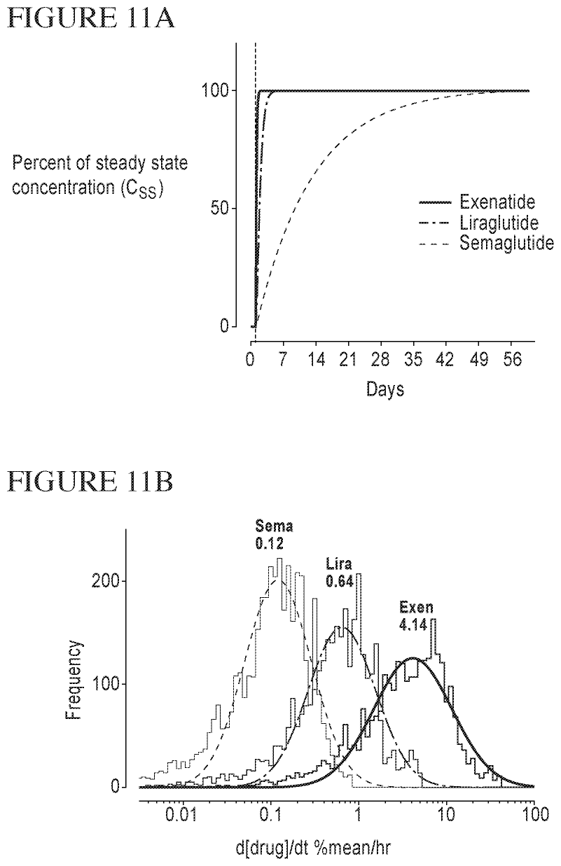

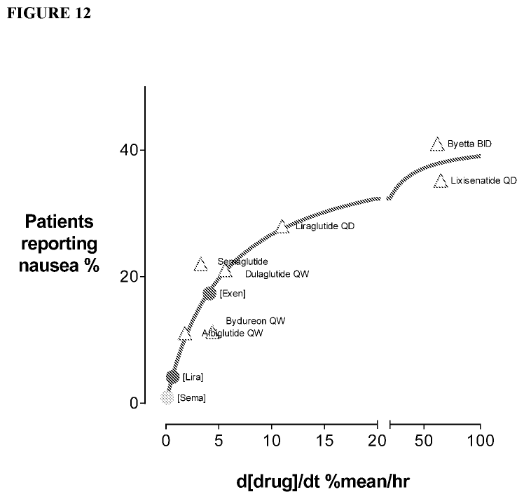

Rate of change of drug concentration, particularly positive rate of change, d[drug]/dt, was derived as the first differential of the plasma concentration profile summed as described above. The d[drug]/dt was expressed with “d[drug]” units reduced to percent of steady state mean concentration of indicated doses, to enable comparisons between agents with differing potencies and pharmacokinetics. The “dt” units were hours. The X-axes in FIGS. 10 and 12 represent the d[drug]/dt thus obtained, in units of “% of steady-state mean (i.e., Cₛₛ) per hour”.

### Example 2. Albumin Binding Assessed by Potency Shift

The following assay was used to test whether GLP-1 receptors were activated by free peptide in solution, but not or less so by peptide that was bound to albumin. The reduction in potency for activation of GLP-1 receptors in the presence of physiologic concentrations of human serum albumin (4% HSA) versus the potency observed with low (0.1%) HSA was used as a measure of the extent of albumin binding. Activation of expressed human GLP-1 receptors was determined as follows:

GLP-1 receptors were transiently expressed in cultured CHO-K1 cells: 1×10⁶ CHO-K1 cells were seeded in T75 flasks and cultured in maintenance media for 48 hours prior to transfecting with GLP-1 receptor expression constructs. For transfection, GLP 1R-containing plasmid DNA was mixed with OptiMEM1 and Lipofectamine 2000 and incubated at room temperature for 20 minutes before addition directly to CHO-K1 cells following a single wash-aspirate step with 1×DPBS+/+. Cells were incubated 48 hours at 37° C., 5% CO₂ to allow for receptor expression.

For peptide treatment of GLP-1R expressing cells, a 10⁻⁴ M stock of each test peptide was diluted to a concentration of 2×10⁻⁷ M in stimulation buffer and then serially diluted with stimulation buffer 10-fold to generate 2× peptide working concentrations ranging from 2×10⁻⁷ M down to 2×10⁻¹⁷ M.

Following aspiration of transfection mixtures, hGLP1R-expressing CHO-K1 cells were washed and aspirated once with 1×DPBS−/−. Cells were dissociated and further incubated before repeated (20×) pipetting to create a uniform suspension, which was then counted using a Cellometer mini (Nexcelom Bioscience). Suspensions were centrifuged at 150×g for five minutes, supernatant removed, and then re-suspended to a density of 1×10⁵ cells per milliliter in stimulation buffer. Aliquots of cell suspensions (500 cells/well) and test peptide in either 0.1% or 4% (or for pilot studies, final concentrations of 0, 0.0125, 0.025, 0.05, 0.1, 0.2, 1, 2 and 4%) of fraction V human serum albumin were added to quadruplicate wells of a 384-well, white opaque OptiPlate (PerkinElmer No. 6007299). Separately, forskolin (system cAMP maximum control) and buffer (system cAMP minimum control) were incubated.

Plates were covered and incubated for 30 minutes at room temperature prior to assessment of cAMP accumulation using the PerkinElmer LANCE Ultra cAMP system. Following addition of tracer and Ulight®-anti-cAMP solution, and a further incubation for 60 minutes in the dark, assay plates were read on a Molecular Devices Flexstation III Multi-Mode plate reader (No. 0310-5627) running SoftMax Pro (version 5.4.6.005).

Test values were normalized to a forskolin-induced cAMP system maximum. Derived cAMP response data were fit to a 3-parameter logistic curve using Prism v6.07 (GraphPad Software, San Diego, Calif.). EC50 values were converted to pEC50 values using the formula: pEC50=−Log(EC50). For each estimated pEC50 value the standard error and R² values were also determined.

Results:

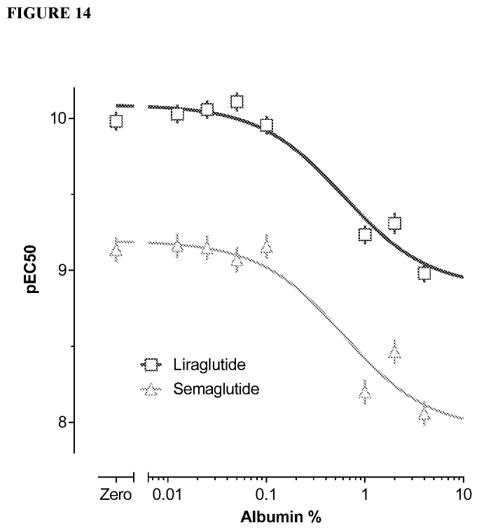

The potency of liraglutide and semaglutide at human GLP-1 receptors depended upon final albumin concentration in the incubation. Potency decreased with increasing albumin concentration, with the mid-range of the change occurring with an albumin concentration of ˜0.6%. See FIG. 14.

Potency shifts were thereafter assessed with albumin concentrations of 0.1% (below which there was no further gain in potency) and 4%, which approximates the concentration found in plasma.

Association of Potency Shift with Mitigation of Nausea:

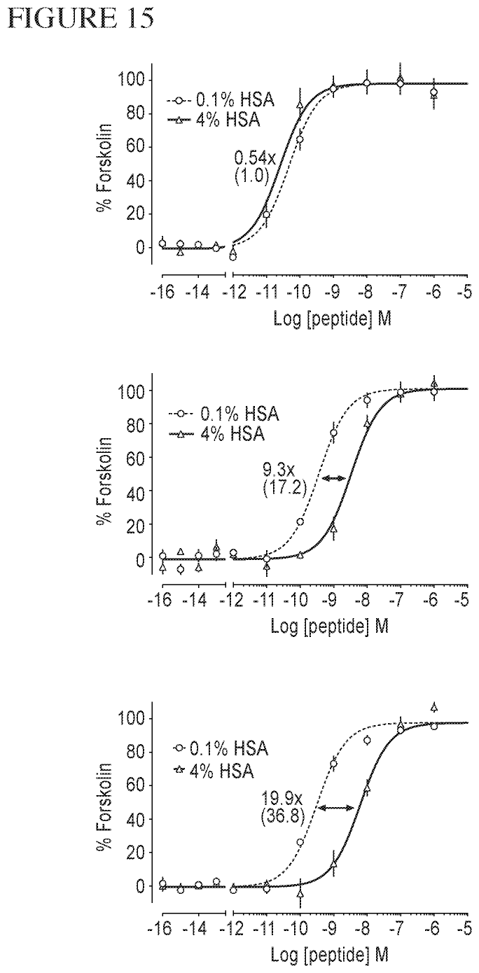

Potency shifts in 4% vs 0.1% albumin were determined for human GLP-1[7-36]NH₂, liraglutide and semaglutide. There was a small (1.8-fold) increase in potency for human GLP-1 [7-36]NH₂ in 4% albumin. In contrast, there was a 9.3-fold decrease in potency for liraglutide, and a 19.9-fold decrease for semaglutide. Relative to the effect observed with GLP-1[7-36]NH₂, these represent 17.2- and 36.8-fold reductions in potency. See FIG. 15.

Changes in human pharmacokinetics and mitigation of nausea at initiation of continuous delivery of a nauseogenic peptide is contemplated for peptides exhibiting significant albumin-mediated potency shift (e.g., 36.8-fold for semaglutide) relative to human GLP-1[7-36]NH₂. Less mitigation of nausea is contemplated for continuous delivery of nauseogenic peptides exhibiting relatively modest albumin-mediated potency shift (e.g., 17.2-fold for liraglutide). Thus, a decrease in potency upon exposure of a nauseogenic peptide to 4% albumin is contemplated to correlate to reductions in the incidence and/or prevalence of nausea upon continuous administration of the nauseogenic peptide according to methods described herein.

### Example 3. Measuring Surrogates for Nausea in Animals

Animal models cannot report nausea. Several models, including rodents, do not vomit, so vomiting in rats is also unavailable as a surrogate of nausea.

Without being bound by theory, nauseogenic compounds are known to mediate their effects via activation of neurons at the area postrema, a brainstem structure that senses nutrients, meal-related peptides and other chemical signals. The same structure mediates the anorectic effects of these same peptide and nutrient stimuli. Dogs, a species that normally vomits, no longer vomit when the area postrema has been surgically ablated. In other species, control of food intake in response to nutrients and meal-related peptides is also impaired when area postrema is ablated. Thus, satiety, anorexia, nausea and vomiting may be considered as a continuum of responses mediated via a common anatomic structure. Alterations in the pattern of one response may reasonably be expected to map alterations in the pattern of another.

The magnitude and pattern of food intake, which is measurable, can thus be used as a surrogate for changes in the dynamics of nauseogenesis.

Method:

Food intake by free-feeding male Long Evans rats is measured continuously. Food (Research Diets D12451i; 45% fat) is contained in dispensers within the BioDAQ system, and its consumption continuously logged as a decrease in food mass on the containing load-cell. Intake data over 4 days are binned into 1-hour epochs to enable comparisons of effect between doses and compounds over different times after administration and throughout the diurnal feeding cycle.

Data can be analyzed as a cumulative effect, or as an instantaneous effect (within a single time “bin”).

Following 1 week of acclimation to the BioDAQ environment, animals are injected subcutaneously with a single dose of an anorectic/nauseogenic agent.
1. As an example of a non-albumin-binding GLP-1 receptor agonist, exenatide is administered in single doses of 0 (vehicle), 0.001, 0.003, 0.01, 0.03, 0.1, and 1.0 mg/kg (n=8/dose group).
2. As an example of an albumin-binding GLP-1 receptor agonist, semaglutide is administered in doses of 0 (vehicle), 0.001, 0.003, 0.01, 0.03, 0.1 and 0.3 mg/kg (n=8/dose group) and food intake and patterns of food intake followed for 5 days.
3. As an example of an anorectic peptide outside the GLP-1 agonist class without significant albumin-binding affinity, pramlintide, an amylin agonist, is administered in doses of 0 (vehicle), 0.01, 0.03, 0.1, 0.3, 1.0 and 3.0 mg/kg (n=8/dose group) and food intake and patterns of food intake followed for 40 hours.
4. As an example of an albumin-binding peptide outside of the GLP-1 agonist class, example 109 from U.S. Pat. No. 9,023,789 B2 (Novo Nordisk), an amylin agonist, is administered in doses of 0 (vehicle), 0.001, 0.003, 0.01, 0.03, 0.1 and 0.3 mg/kg (n=8/dose group) and food intake and patterns of food intake followed for 5 days.

To illustrate differences in pharmacodynamic profiles, that map to a benefit in reduction of nausea, these agents are delivered continuously via ALzet 2ML2 mini-osmotic pumps. After surgical implantation, animals were returned to the BioDAQ environment for continuous measurement of ingestive behavior. Pumps are loaded with formulation designed to deliver agents described in (1)-(4) above at infusion rates of 0 (vehicle), 0.001, 0.003, 0.01, 0.03, 0.1 and 0.3 mg/kg/day.

### Example 4. Pharmacokinetic Methods

Pharmacokinetics of peptides are studied in male Sprague Dawley rats (Charles River Laboratories, Raleigh) previously implanted with a vascular access ports (Instech) implanted in femoral and jugular veins.

To characterize intravenous pharmacokinetics, peptide is infused intravenously for 1 hour at a total dose of 0.033 mg/kg. Samples of 250 μL are taken from the jugular port at t=0.25, 0.5, 0.75, 1*, 1.17, 1.33, 1.5, 2, 3, 5, 9, 24 hours. Sample is mixed with 25 μL K₂EDTA, protease inhibitor cocktail. The 1 hour sample is taken before cessation of intravenous peptide infusion. There are n=3 animals per group.

To characterize subcutaneous pharmacokinetics, peptide is injected subcutaneously at a bolus dose of 0.3 mg/kg (2.5 mL/kg). Samples are taken at 0.083, 0.167, 0.25, 0.5, 1, 2, 4, 8, 24 and 30 hours after injection. There are n=3 animals per group.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A method for treating a subject, comprising
contacting the subject with a drug delivery device comprising a nauseogenic compound, wherein the drug delivery device administers the nauseogenic compound to the subject, and the contacting occurs after an administration of the drug delivery device comprising the nauseogenic compound to a human patient population during a first clinical trial;
wherein
less than 10% of the human patient population, to whom the drug delivery device comprising the nauseogenic compound was administered, reported having nausea and/or vomiting during the first clinical trial.

2. The method of claim 1, wherein less than 5% of the human patient population reported having nausea and/or vomiting during the first clinical trial.

3. A method for treating a subject, comprising
contacting the subject with a drug delivery device comprising a nauseogenic compound and the drug delivery device administers the nauseogenic compound to the subject,
wherein
incidence of nausea and/or vomiting is 10% or less during a first clinical trial regarding administration of the drug delivery device comprising a continuous dose of the nauseogenic compound to a first human patient population; and
incidence of nausea and/or vomiting is 15% or greater during a second clinical trial regarding administration of an injectable or oral dose of the nauseogenic compound to a second human patient population.

4. A method for treating a subject, comprising
contacting the subject with a drug delivery device comprising a nauseogenic compound and the drug delivery device administers the nauseogenic compound to the subject,
wherein
incidence of nausea and/or vomiting, reported as a percentage of a first human patient population, during a first clinical trial regarding administration of the drug delivery device comprising a continuous dose of the nauseogenic compound to the first human patient population, is reduced by at least 20% relative to incidence of nausea and/or vomiting, reported as a percentage of a second human patient population, during a second clinical trial regarding an administration of an injectable or an oral dose of the nauseogenic compound to the second human patient population.

5. The method of any one of the preceding claims, for treating type-2 diabetes in a subject.

6. The method of any one of the preceding claims, wherein the nauseogenic compound is a nauseogenic peptide.

7. The method of any one of the preceding claims, wherein the nauseogenic compound is a long-acting nauseogenic peptide.

8. The method of any one of the preceding claims, for treating a subject for type-2 diabetes, comprising contacting the subject with an implantable osmotic drug delivery device comprising a long-acting nauseogenic peptide.

9. The method of any one of claims 1-4, wherein the long-acting nauseogenic peptide is selected from GLP-1 receptor agonist, PYY analog, amylin agonist, CGRP analog, or neurotensin analog.

10. The method of any one of the preceding claims, wherein the nauseogenic compound is a GLP-1 receptor agonist.

11. The method of claim 10, wherein the long-acting GLP-1 receptor agonist is exenatide dispersed in a biocompatible polymer (Bydureon®), semaglutide (Ozempic®), liraglutide (Victoza®), albiglutide (Tanzeum®), or dulaglutide (Trulicity®).

12. The method of claim 11, wherein the long-acting GLP-1 receptor agonist is semaglutide.

13. The method of claim 11, wherein the long-acting GLP-1 receptor agonist is liraglutide.

14. The method of claim 11, wherein the long-acting GLP-1 receptor agonist is albiglutide.

15. The method of claim 11, wherein the long-acting GLP-1 receptor agonist is dulaglutide.

16. The method of claim 11, wherein the long-acting GLP-1 receptor agonist is exenatide dispersed in a biocompatible polymer.

17. The method of any preceding claim, wherein the drug delivery device administers the nauseogenic compound to the subject,
during the first 24 hours following initiation of administration, less than or equal to 90% of mean steady state concentration (Cₛₛ) of the nauseogenic compound is attained in the plasma of the subject; and
once Cₛₛ is attained, Cₛₛ of the nauseogenic compound is maintained in the plasma of the subject for at least two weeks.

18. A method for treating a subject, comprising contacting the subject with a drug delivery device comprising a dose of a nauseogenic compound,
wherein
the drug delivery device administers the nauseogenic compound to the subject,
during the first 24 hours following initiation of administration, less than or equal to 90% of mean steady state concentration (Cₛₛ) of the nauseogenic compound is attained in the plasma of the subject; and
once Cₛₛ is attained, Cₛₛ of the nauseogenic compound is maintained in the plasma of the subject for at least two weeks.

19. The method of claim 17 or 18, wherein incidence of nausea is reduced in the subject during treatment.

20. The method of any one of claims 17-19, wherein incidence of nausea is less than 10% of subjects treated.

21. The method of any one of claims 17-20, wherein incidence of nausea is less than 5% of subjects treated.

22. The method of any one of claims 17-21, wherein during the first 2 days following initiation of administration, less than or equal to 90% of mean steady state concentration (Cₛₛ) of the nauseogenic compound is attained in the plasma of the subject.

23. The method of any one of claims 17-22, wherein during the first 7 days following initiation of administration, less than or equal to 90% of mean steady state concentration (Cₛₛ) of the nauseogenic compound is attained in the plasma of the subject.

24. The method of any one of claims 17-23, wherein during the first 14 days following initiation of administration, less than or equal to 90% of mean steady state concentration (Cₛₛ) of the nauseogenic compound is attained in the plasma of the subject.

25. The method of any one of claims 17-24, wherein initial concentration (C_{I}) in plasma of the nauseogenic compound, during the first 12 hours following initiation of administration, is less than or equal to 25% of mean steady state concentration (Cₛₛ) in plasma of the nauseogenic compound that will be attained in the subject.

26. The method of any one of claims 17-25, wherein d[nauseogenic compound]/dt is less than 4% of the mean steady state concentration (Cₛₛ) of the nauseogenic compound per hour.

27. The method of any one of claims 17-26, wherein d[nauseogenic compound]/dt is less than 2% of the mean steady state concentration (Cₛₛ) of the nauseogenic compound per hour.

28. The method of any one of claims 17-27, wherein d[nauseogenic compound]/dt is less than 1% of the mean steady state concentration (Cₛₛ) of the nauseogenic compound per hour.

29. The method of any one of claims 17-28, wherein d[nauseogenic compound]/dt is less than 0.5% of the mean steady state concentration (Cₛₛ) of the nauseogenic compound per hour.

30. The method of any one of claims 17-29, wherein d[nauseogenic compound]/dt is less than 0.25% of the mean steady state concentration (Cₛₛ) of the nauseogenic compound per hour.

31. The method of any one of claims 17-30, wherein
d[nauseogenic compound]/dt is less than 4% of the mean steady state concentration (Cₛₛ) of the nauseogenic compound per hour; and
less than or equal to 90% of mean steady state concentration (Cₛₛ) of the nauseogenic compound is attained in the plasma of the subject during the first 7 days following administration.

32. The method of any one of claims 17-31, without incurring substantial peak-trough fluctuations in plasma concentration of the nauseogenic compound.

33. The method of any one of claims 17-32, wherein a maximum steady state concentration Cₘₐₓ of nauseogenic compound does not substantially exceed the mean steady state concentration (Cₛₛ) of the nauseogenic compound.

34. The method of any one of claims 17-33, wherein the mean steady state concentration (Cₛₛ) of the nauseogenic compound, once reached in the subject, is substantially maintained for at least one month.

35. The method of any one of claims 1-34, wherein the nauseogenic compound has an elimination half-life (t_{1/2}) in humans of 1 day to 14 days.

36. The method of any one of claims 1-35, wherein the nauseogenic compound has an elimination half-life (t_{1/2}) in humans of 6 days to 10 days.

37. The method of any one of claims 1-36, wherein the device is an implantable drug delivery device.

38. The method of any one of claims 1-37, wherein the device is an implantable osmotic drug delivery device.

39. The method of claim 38, wherein contacting comprises subdermal placement of the implantable osmotic drug delivery device.

40. The method of claim 38, wherein the implantable osmotic drug delivery device administers a continuous dose of the nauseogenic compound.

41. The method of any one of claims 1-40, wherein the nauseogenic compound is a long-acting nauseogenic peptide.

42. The method of any one of claims 1-36, wherein the device is a non-implantable delivery device.

43. The method of claim 42, wherein contacting comprises affixing the non-implantable delivery device to an outer surface of the patient's skin.

44. The method of any one of claims 1-43, for treating diabetes in a subject.

45. The method of any one of claims 1-44, for treating type-2 diabetes in a subject.

46. The method of any one of claims 1-45, wherein the drug delivery device comprises a solid suspension of the nauseogenic compound.

47. The method of any one of claims 1-46, wherein the drug delivery device comprises an anhydrous formulation of the nauseogenic compound.

48. The method of any one of claims 1-47, wherein the nauseogenic compound is a long-acting nauseogenic peptide having a binding affinity to its intended receptor that is decreased 20-50 fold in the presence of 4% human serum albumin when comparing the binding affinity in the presence of very low concentration 0.1% of human serum albumin.

49. The method of any one of claims 1-48, wherein the nauseogenic compound is a long-acting nauseogenic peptide having an apparent K_{D} for association with albumin not greater than 1 micromole/liter.

50. The method of any one of claims 1-49, wherein the nauseogenic compound is a long-acting nauseogenic peptide having an off rate for dissociation of nauseogenic compound from albumin not greater than 0.002/sec.

51. The method of any one of claims 1-50, wherein the nauseogenic compound is an acylated long-acting GLP-1 receptor agonist that binds human serum albumin (HSA) and exhibits an albumin-mediated potency decrease of 10-25 fold in the presence of 4% human serum albumin relative its potency in the presence of very low concentration 0.1% of human serum albumin.

52. The method of any one of claims 1-51, wherein the nauseogenic compound is an acylated long-acting GLP-1 receptor agonist that binds human serum albumin (HSA) and exhibits an albumin-mediated potency shift 20-50 fold relative to potency shift for human GLP-1 [7-36]NH₂.

53. The method of any one of claims 1-52, wherein the nauseogenic compound is a nauseogenic peptide.

54. The method of any one of claims 1-53, wherein the nauseogenic compound is a long-acting nauseogenic peptide.

55. A method for treating a subject for type-2 diabetes, comprising contacting the subject with an implantable osmotic drug delivery device comprising a long-acting nauseogenic peptide.

56. The method of any one of claims 1-54, wherein the nauseogenic compound or long-acting nauseogenic peptide comprises a lipophilic group, optionally bound to the peptide via a spacer.

57. The method of any one of claims 1-56, wherein the nauseogenic compound or long-acting nauseogenic peptide has a t_{1/2} of at least about 24 hours in humans following subcutaneous administration.

58. The method of any one of claims 17-57, wherein the long-acting nauseogenic peptide is selected from GLP-1 receptor agonist, PYY analog, amylin agonist, CGRP analog, or neurotensin analog.

59. The method of any one of claims 17-58, wherein the long-acting nauseogenic peptide is selected from GLP-1 receptor agonist, PYY analog, amylin agonist, CGRP analog, or neurotensin analog, each of which comprises a lipophilic group, optionally bound to the peptide via a spacer.

60. The method of any one of claims 17-59, wherein the long-acting nauseogenic peptide is a GLP-1 receptor agonist.

61. The method of claim 60, wherein the long-acting GLP-1 receptor agonist is selected from any of the compounds of Formula I, Formula II, Formula III, Formula IV, and Formula V.

62. The method of claim 60, wherein the long-acting GLP-1 receptor agonist is selected from any of the compounds of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10.

63. The method of claim 60, wherein the long-acting GLP-1 receptor agonist is exenatide dispersed in a biocompatible polymer (Bydureon®), semaglutide (Ozempic®), liraglutide (Victoza®), albiglutide (Tanzeum®), or dulaglutide (Trulicity®).

64. The method of claim 63, wherein the long-acting GLP-1 receptor agonist is semaglutide.

65. The method of claim 63, wherein the long-acting GLP-1 receptor agonist is liraglutide.

66. The method of claim 63, wherein the long-acting GLP-1 receptor agonist is albiglutide.

67. The method of claim 63, wherein the long-acting GLP-1 receptor agonist is dulaglutide.

68. The method of claim 63, wherein the long-acting GLP-1 receptor agonist is exenatide dispersed in a biocompatible polymer.

69. An apparatus comprising a drug delivery device and a nauseogenic compound that, upon being contacted with a subject, provides administration of a dose of the nauseogenic compound to the subject;
wherein
during the first 24 hours following initiation of administration, less than or equal to 90% of mean steady state concentration (Cₛₛ) of the nauseogenic compound is attained in the plasma of the subject; and
once Cₛₛ is attained, Cₛₛ of the nauseogenic compound is maintained in the plasma of the subject for at least two weeks.


SEQUENCE LISTING (12 sequences)

SEQ ID NO: 1 - protein, 39 aa, Heloderma suspectum
HGEGTFTSDLSKQMEEEAVRLFIEWLKNGGPSSGAPPPS

SEQ ID NO: 2 - protein, 44 aa, Artificial Sequence
  source: /note=“Description of Artificial Sequence Synthetic Lixisenatide, the short-acting GLP-1 receptor agonist which is a synthetic analog of exenatide”
HGEGTFTSDLSKQMEEEAVRLFIEWLKNGGPSSGAPPSKKKKKK

SEQ ID NO: 3 - protein, 31 aa, Artificial Sequence
  source: /note=“Description of Artificial Sequence Synthetic polypeptide”
HAEGTFTSDVSSYLEGQAAKEFIAWLVRGRG

SEQ ID NO: 4 - protein, 33 aa, Artificial Sequence
  source: /note=“Description of Artificial Sequence Synthetic polypeptide”
  VARIANT (1)..(1): /replace=“a modified amino acid” or “ ”
  VARIANT (2)..(2): /replace=“Gly” or “Ser” or “Thr” or “Leu” or “Ile” or “Val” or “Glu” or “Asp”
  VARIANT (12)..(12): /replace=“Ala” or “Gly” or “Thr” or “Leu” or “Ile” or “Val” or “Glu” or “Asp” or “Lys”
  VARIANT (13)..(13): /replace=“Phe” or “Trp” or “Glu” or “Asp” or “Lys”
  VARIANT (14)..(14): /replace=“Ala” or “Gly” or “Ser” or “Thr” or “Leu” or “Ile” or “Val” or “Glu” or “Asp” or “Lys”
  VARIANT (15)..(15): /replace=“Asp” or “Lys”
  VARIANT (16)..(16): /replace=“Ala” or “Ser” or “Thr” or “Leu” or “Ile” or “Val” or “Glu” or “Asp” or “Lys”
  VARIANT (17)..(17): /replace=“Asn” or “Arg” or “Glu” or “Asp” or “Lys”
  VARIANT (18)..(18): /replace=“Gly” or “Ser” or “Thr” or “Leu” or “Ile” or “Val” or “Arg” or “Glu” or “Asp” or “Lys”
  VARIANT (19)..(19): /replace=“Gly” or “Ser” or “Thr” or “Leu” or “Ile” or “Val” or “Glu” or “Asp” or “Lys”
  VARIANT (20)..(20): /replace=“Arg” or “Gln” or “Glu” or “Asp” or “His”
  VARIANT (21)..(21): /replace=“Asp” or “Lys”
  VARIANT (24)..(24): /replace=“Gly” or “Ser” or “Thr” or “Leu” or “Ile” or “Val” or “Glu” or “Asp” or “Lys”
  VARIANT (25)..(25): /replace=“Phe” or “Tyr” or “Glu” or “Asp” or “Lys”
  VARIANT (26)..(26): /replace=“Gly” or “Ala” or “Ser” or “Thr” or “Ile” or “Val” or “Glu” or “Asp” or “Lys”
  VARIANT (27)..(27): /replace=“Gly” or “Ala” or “Ser” or “Thr” or “Leu” or “Ile” or “Glu” or “Asp” or “Lys”
  VARIANT (28)..(28): /replace=“Arg” or “Glu” or “Asp” or “His”
  VARIANT (29)..(29): /replace=“Ala” or “Ser” or “Thr” or “Leu” or “Ile” or “Val” or “Glu” or “Asp” or “Lys”
  VARIANT (30)..(30): /replace=“Lys” or “Glu” or “Asp” or “His”
  VARIANT (31)..(31): /replace=“Ala” or “Ser” or “Thr” or “Leu” or “Ile” or “Val” or “Glu” or “Asp” or “Lys” or “ ”
  VARIANT (32)..(32): /replace=“Lys” or “Glu” or “Asp” or “His” or “ ”
  VARIANT (33)..(33): /replace=“ ”
  MISC_FEATURE (1)..(33): /note=“Variant residues given in the sequence have no preference with respect to those in the annotations for variant positions”
  source: /note=“See specification as filed for detailed description of substitutions and preferred embodiments”
HAEGTFTSDVSSYLEGQAAKEFIAWLVKGRGRR

SEQ ID NO: 5 - protein, 31 aa, Artificial Sequence; identical to SEQ ID NO: 3
  source: /note=“Description of Artificial Sequence Synthetic polypeptide”
  source: /note=“See specification as filed for detailed description of substitutions and preferred embodiments”

SEQ ID NO: 6 - protein, 31 aa, Artificial Sequence
  source: /note=“Description of Artificial Sequence Synthetic polypeptide”
  MOD_RES (2)..(2): Aib
HXEGTFTSDVSSYLEGQAAKEFIAWLVRGRG

SEQ ID NO: 7 - protein, 32 aa, Artificial Sequence
  source: /note=“Description of Artificial Sequence Synthetic polypeptide”
  VARIANT (1)..(1): /replace=“D-his” or “desamino-his” or “2-amino-his” or “beta- hydroxy-his” or “homohis” or “Nalpha-acetyl-his” or “a- fluoromethyl-his” or “a-methyl-his” or “3-pyridylalanine” or “2-pyridylalanine” or “4-pyridylalanine”
  VARIANT (2)..(2): /replace=“Val” or “Leu” or “Ile or ”Lys“ or ”Aib“ or ”(1-aminocyclopropyl) carboxylic acid“ or ”(1-aminocyclobutyl)“ carboxylic acid” or “(1-aminocyclopentyl) carboxylic acid” or “(1-aminocyclohexyl) carboxylic acid” or “(1-aminocycloheptyl)
  VARIANT (2)..(2): Continued from above;/replace=”carboxylic acid“ or ”(1- aminocyclooctyl) carboxylic acid“
  VARIANT (10)..(10): /replace=”Leu“
  VARIANT (12)..(12): /replace=”Lys“ or ”Arg“
  VARIANT (13)..(13): /replace=”Gln“
  VARIANT (14)..(14): /replace=”Met“
  VARIANT (16)..(16): /replace=”Glu“ or ”Aib“
  VARIANT (17)..(17): /replace=”Glu“ or ”Lys“ or ”Arg“
  VARIANT (19)..(19): /replace=”Val“
  VARIANT (21)..(21): /replace=”Leu“
  VARIANT (24)..(24): /replace=”Glu“ or ”Arg“
  VARIANT (27)..(27): /replace=”Lys“
  VARIANT (28)..(28): /replace=”Glu“ or ”Asn“ or ”Arg“
  VARIANT (29)..(29): /replace=”Aib“
  VARIANT (30)..(30): /replace=”Gly“ or ”Lys“ or ” “
  VARIANT (31)..(31): /replace=”Ala“ or ”Glu“ or ”Pro“ or ”Lys“ or ” “
  VARIANT (32)..(32): /replace=”Ser“ or ” “
  MISC_FEATURE (1)..(32): /note=”Variant residues given in the sequence have no preference with respect to those in the annotations for variant positions“
  source: /note=”See specification as filed for detailed description of substitutions and preferred embodiments“
HGEGTFTSDVSSYLEGQAAKEFIAWLVKGRGK

SEQ ID NO: 8 - protein, 31 aa, Homo sapiens
HAEGTFTSDVSSYLEGQAAKEFIAWLVKGRG

SEQ ID NO: 9 - protein, 645 aa, Artificial Sequence
  source: /note=”Description of Artificial Sequence Synthetic polypeptide“
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 10 - protein, 275 aa, Artificial Sequence
  source: /note=”Description of Artificial Sequence Synthetic polypeptide“
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 14 aa, Artificial Sequence
  source: /note=”Description of Artificial Sequence Synthetic linker“
GGGGGGGSGGGGSG

SEQ ID NO: 12 - protein, 16 aa, Artificial Sequence
  source: /note=”Description of Artificial Sequence Synthetic peptide“
GGGGSGGGGSGGGGSA